(19) United States
(12) Patent Application Publication
(10) Pub. No.: US 2009/0093468 A1
(43) Pub. Date: Apr. 9, 2009

(54) Peptide Deformylase Inhibitors

(76) Inventors: Patrizia Cali, Copenhagen S (DK); Anders Hjelmencrantz, Malmo (SE); Lars Naerum, Hellerup (DK)

Correspondence Address: EDWARDS ANGELL PALMER & DODGE LLP, P.O. BOX 55874, BOSTON, MA, 02205, US

(21) Appl. No.: 10/594,360

(22) PCT Filed: Mar. 22, 2005
(86) PCT No.: PCT/DK2005/000201

§ 371 Date: Nov. 13, 2008

Related U.S. Application Data
(60) Provisional application No. 60/557,985, filed on Mar. 31, 2004.

(30) Foreign Application Priority Data
Mar. 26, 2004 (DK) PA 2004 00489

(51) Int. Cl. A61K 31/5415; C07D 279/16; A61P 31/04

(52) U.S. Cl. 514/224.2; 544/52

(57) ABSTRACT

Benzothiazine compounds of the general formula (I) and pharmaceutically acceptable salts or esters thereof are peptide deformylase inhibitors useful in the treatment or prevention of infections and other diseases in which peptide deformylases are involved, especially in the treatment of bacterial and parasitic infections, for example infections fully or partly caused by microorganisms belonging to Staphylococcus, Enterococcus, Streptococcus, Haemophilus, Moraxella, Escherichia, Mycobacterium, Mycoplasma, Pseudomonas, Chlamydia, Rickettsia, Klebsiella, Shigella, Salmonella, Bordetella, Clostridium, Helicobacter, Campylobacter, Legionella or Neisseria.

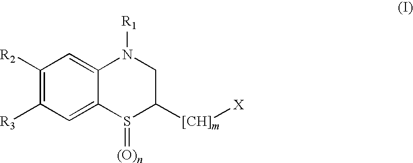

The present invention relates to novel enzyme inhibitors, more specifically to inhibitors of polypeptide deformylase useful in the treatment/prevention of infections and other diseases in which polypeptide deformylases are involved, especially in the treatment of bacterial and parasitic infections. More specifically the invention relates to benzothiazines capable of inhibiting bacterial peptide deformylase, also known as PDF, an enzyme that catalyzes the deformylation of formyl-L-methionyl peptides.

### BACKGROUND OF THE INVENTION

Peptide deformylase (EC 3.4.1.88), also known as PDF, is an enzyme that catalyzes the deformylation of formyl-L-methionyl peptides. PDF removes the formyl group from the N-terminal Met of newly synthesized proteins, i.e. catalyzes the conversion of formyl-L-methionyl peptide to methionyl peptide (Adams and Capecchi, 1966; Adams, 1968). PDF is essential to bacteria, and bacterial peptide deformylase (PDF) is now widely recognised as an attractive target for antibacterial chemotherapy (Giglione et al., 2000; Giglione and Meinnel, 2001; Pei 2001; Yuan et al., 2001; Clements et al., 2002). Deformylation is a crucial step in bacterial protein biosynthesis and PDF is an essential ingredient in bacterial growth, with the gene encoding PDF present in all sequenced pathogenic bacterial genomes.

Novel antibacterial compounds are urgently needed due to the growing resistance exhibited by both Gram-negative and Gram-positive bacteria and other microorganisms. Traditional antibiotics have targeted pathways unique to bacterial replication and maintenance. However, new pathways are not being targeted in a manner that outpaces the growth of bacterial resistance. Thus, novel compounds and strategies are greatly needed that can be used to eradicate resistant bacteria.

### SUMMARY OF THE INVENTION

The present invention relates to compounds of the general formula (I)

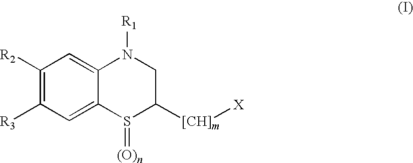

or a pharmaceutically acceptable salt or ester thereof,
wherein R₁, R₂, R₃, n, m and X are as defined in the detailed part of this description.

It is contemplated that the compounds of the invention are useful for the treatment of infections caused by bacteria or parasites. It is especially contemplated that the compounds of the present invention are useful for the treatment of infections fully or partly caused by Gram-positive or Gram-negative bacteria such as Escherichia coli and Staphylococcus aureus or by a parasite such as Plasmodium falciparum.

It is an object of the invention to provide novel compounds having pharmacological activity as inhibitors of PDF.

Further objects will become apparent from the following description.

### DETAILED DESCRIPTION OF THE INVENTION

### Definitions

The terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

The term “peptide deformylase” or “PDF” as used herein is intended to mean peptide deformylase (EC 3.4.1.88) also known as PDF, which catalyzes the conversion of the N-terminal formyl-L-methionyl peptide to methionyl peptide in newly synthesized proteins. The term “treatment” is defined as the management and care of a patient for the purpose of combating the disease, condition, or disorder and includes the administration of a compound of the present invention to prevent the onset of the symptoms or the complications, or alleviating the symptoms or the complications, or eliminating the disease, condition, or disorder.

As used herein, alone or in combination, the term “C₁₋₆ alkyl” denotes a straight or branched, saturated hydrocarbon chain having from one to six carbon atoms. C₁₋₆ alkyl groups include, but are not limited to, methyl, ethyl, n-propyl, iso-propyl, n-butyl, iso-butyl, sec-butyl, tert-butyl, n-pentyl, iso-pentyl, 2-methylbutyl, 3-methylbutyl, n-hexyl, iso-hexyl, 4-methylpentyl, neopentyl, 2,2-dimethylpropyl and the like.

As used herein, alone or in combination, the term “C₂₋₆ alkenyl” denotes a straight or branched, unsaturated hydrocarbon chain having from two to six carbon atoms and at least one double bond. C₂₋₆ alkenyl groups include, but are not limited to, vinyl, 1-propenyl, allyl, iso-propenyl, n-butenyl, n-pentenyl, n-hexenyl and the like.

The term “C₁₋₆ alkoxy” in the present context designates a group —O—C₁₋₆ alkyl used alone or in combination, wherein C₁₋₆ alkyl is as defined above. Examples of linear alkoxy groups are methoxy, ethoxy, propoxy, butoxy, pentoxy and hexoxy. Examples of branched alkoxy are iso-propoxy, sec-butoxy, tert-butoxy, iso-pentoxy and iso-hexoxy. Examples of cyclic alkoxy are cyclopropyloxy, cyclobutyloxy, cyclopentyloxy and cyclohexyloxy.

The term “C₃₋₁₀ cycloalkyl” as used herein denotes a radical of one or more saturated mono-, bi-, tri- or spirocyclic hydrocarbon having from three to ten carbon atoms. Examples include, but are not limited to, cyclopropyl, cyclobutyl, cyclopentyl, cyclohexyl, cycloheptyl, cyclooctyl, cyclononyl, cyclodecyl, bicyclo[3.2.1]octyl, spiro[4.5]decyl, norpinyl, norbonyl, norcaryl, adamantyl and the like.

The term “C₃₋₇ heterocycloalkyl” as used herein denotes a radical of a totally saturated heterocycle like a cyclic hydrocarbon containing one or more heteroatoms selected from nitrogen, oxygen and sulphur independently in the cycle. Examples of heterocycles include, but are not limited to, pyrrolidine (1-pyrrolidine, 2-pyrrolidine, 3-pyrrolidine, 4-pyrrolidine, 5-pyrrolidine), pyrazolidine (1-pyrazolidine, 2-pyrazolidine, 3-pyrazolidine, 4-pyrazolidine, 5-pyrazolidine), imidazolidine (1-imidazolidine, 2-imidazolidine, 3-imidazolidine, 4-imidazolidine, 5-imidazolidine), thiazolidine (2-thiazolidine, 3-thiazolidine, 4-thiazolidine, 5-thiazolidine), piperidine (1-piperidine, 2-piperidine, 3-piperidine, 4-piperidine, 5-piperidine, 6-piperidine), piperazine (1-piperazine, 2-piperazine, 3-piperazine, 4-piperazine, 5-piperazine, 6-piperazine), morpholine (2-morpholine, 3-morpholine, 4-morpholine, 5-morpholine, 6-morpholine), thiomorpholine (2-thiomorpholine, 3-thiomorpholine, 4-thiomorpholine, 5-thiomorpholine, 6-thiomorpholine), 1,2-oxathiolane (3-(1,2-oxathiolane), 4-(1,2-oxathiolane), 5-(1,2-oxathiolane)), 1,3-dioxolane (2-(1,3-dioxolane), 3-(1,3-dioxolane), 4-(1,3-dioxolane)), tetrahydropyrane (2-tetrahydropyrane, 3-tetrahydropyrane, 4 tetrahydropyrane, 5-tetrahydropyrane, 6-tetrahydropyrane), hexahydropyradizine, (1-(hexahydropyradizine), 2-(hexahydropyradizine), 3-(hexahydropyradizine), 4-(hexahydropyradizine), 5-(hexahydropyradizine), 6-(hexahydropyradizine)).

The term “C₁₋₆alkyl-C₃₋₁₀cycloalkyl” as used herein refers to a cycloalkyl group as defined above attached through an alkyl group as defined above having the indicated number of carbon atoms.

The term “C₁₋₆alkyl-C₃₋₇heterocycloalkyl” as used herein refers to a heterocycloalkyl group as defined above attached through an alkyl group as defined above having the indicated number of carbon atoms.

The term “aryl” as used herein is intended to include carbocyclic aromatic ring systems. Aryl is also intended to include the partially hydrogenated derivatives of the carbocyclic systems enumerated below.

The term “heteroaryl” as used herein includes heterocyclic unsaturated ring systems containing one or more heteroatoms selected among nitrogen, oxygen and sulphur, such as furyl, thienyl, pyrrolyl, and is also intended to include the partially hydrogenated derivatives of the heterocyclic systems enumerated below.

The terms “aryl” and “heteroaryl” as used herein refers to an aryl, which can be optionally unsubstituted or mono-, di- or tri substituted, or a heteroaryl, which can be optionally unsubstituted or mono-, di- or tri substituted. Examples of “aryl” and “heteroaryl” include, but are not limited to, phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), phenanthrenyl, fluorenyl, pentalenyl, azulenyl, biphenylenyl, thiophenyl (1-thienyl, 2-thienyl), furyl (1-furyl, 2-furyl), furanyl, thiophenyl, isoxazolyl, isothiazolyl, 1,2,3-triazolyl, 1,2,4-triazolyl, pyranyl, pyridazinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuranyl, benzothiophenyl (thianaphthenyl), indolyl, oxadiazolyl, isoxazolyl, quinazolinyl, fluorenyl, xanthenyl, isoindanyl, benzhydryl, acridinyl, benzisoxazolyl, purinyl, quinazolinyl, quinolizinyl, quinolinyl, isoquinolinyl, quinoxalinyl, naphthyridinyl, phteridinyl, azepinyl, diazepinyl, pyrrolyl (2-pyrrolyl), pyrazolyl (3-pyrazolyl), 5-thiophene-2-yl-2H-pyrazol-3-yl, imidazolyl (1-imidazolyl, 2-imidazolyl, 4-imidazolyl, 5-imidazolyl), triazolyl (1,2,3-triazol-1-yl, 1,2,3-triazol-2-yl, 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5 thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), isoquinolyl (1-isoquinolyl, 3-isoquinolyl, 4-isoquinolyl, 5-isoquinolyl, 6-isoquinolyl, 7-isoquinolyl, 8-isoquinolyl), quinolyl (2-quinolyl, 3-quinolyl, 4-quinolyl, 5-quinolyl, 6-quinolyl, 7-quinolyl, 8-quinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydrobenzo[b]furanyl)-5-(2,3-dihydro-benzo[b]furanyl), 6-(2,3-dihydro-benzo[b]furanyl), 7-(2,3-dihydro-benzo[b]furanyl)), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl)), indolyl (1-indolyl, 2-indolyl, 3-indolyl, 4-indolyl, 5-indolyl, 6-indolyl, 7-indolyl), indazolyl (1-indazolyl, 2-indazolyl, 3-indazolyl, 4-indazolyl, 5-indazolyl, 6-indazolyl, 7-indazolyl), benzimidazolyl, (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl). Non-limiting examples of partially hydrogenated derivatives are 1,2,3,4-tetrahydronaphthyl, 1,4-dihydronaphthyl, pyrrolinyl, pyrazolinyl, indolinyl, oxazolidinyl, oxazolinyl, oxazepinyl and the like.

The term “C₁₋₆ alkylaryl” as used herein refers to an aryl group as defined above attached through a C₁₋₆ alkyl group as defined above having one, two, three, four, five or six carbon atoms; the C₁₋₆ alkylaryl can optionally be unsubstituted or substituted.

The term “C₁₋₆ alkylheteroaryl” as used herein refers to a heteroaryl group as defined above attached through a C₁₋₆ alkyl group as defined above having one, two, three, four, five or six carbon atoms; the C₁₋₆ alkylaryl can optionally be unsubstituted or substituted.

The term “thioC₁₋₆-alkyl” in the present context designates a group —S—C₁₋₆alkyl wherein C₁₋₆ alkyl is as defined above. Representative examples include, but are not limited to, methylthio, ethylthio, n-propylthio, isopropylthio, butylthio, isobutylthio, sec-butylthio, tert-butylthio, n-pentylthio, isopentylthio, neopentylthio, tert-pentylthio, n-hexylthio, isohexylthio and the like.

The term “C₁₋₆-alkylmercapto” in the present context designates a group —C₁₋₆alkyl-SH wherein C₁₋₆alkyl is as defined above. Representative examples include, but are not limited to, mercapto methyl (i.e. —CH₂—SH), mercapto ethyl, mercapto n-propyl, mercapto isopropyl, mercapto butyl, mercapto isobutyl, mercapto sec-butyl, mercapto tert-butyl, mercapto n-pentyl, mercapto isopentyl, mercapt neopentylo, mercapto tert-pentyl, mercapto n-hexyl, mercapto isohexyl and the like.

The term “C₁₋₆alkylhydroxyl” in the present context designates a group —C₁₋₆-alkyl-OH wherein C₁₋₆alkyl is as defined above. Representative examples include, but are not limited to, methylhydroxy, ethylhydroxy, n-propylhydroxy, isopropylhydroxy, butylhydroxy, isobutylhydroxy, sec-butylhydroxy, tert-butylhydroxy, n-pentylhydroxy, isopentylhydroxy, neopentylhydroxy, tert-pentylhydroxy, n-hexylhydroxy, isohexylhydroxy and the like.

The term “C₁₋₆-alkylamino” in the present context designates a group —C₁₋₆-alkyl-NH₂ wherein C₁₋₆-alkyl is as defined above. Representative examples include, but are not limited to, methylamino (i.e. —CH₂—NH₂), ethylamino, n-propylamino, isopropylamino, butylamino, isobutylamino, sec-butylamino, tert-butylamino, n-pentylamino, isopentylamino, neopentylamino, tert-pentylamino, n-hexylamino, isohexylamino and the like.

The term “alkylamino-C₁₋₆alkyl” in the present context designates a group C₁₋₆-alkyl-NH—C₁₋₆-alkyl wherein C₁₋₆-alkyl is as defined above. Representative examples include, but are not limited to, methylamino methyl, ethylamino methyl (i.e —CH₂—NH—C₂H₅), n-propylamino methyl, isopropylamino methyl, butylamino methyl, isobutylamino methyl, sec-butylamino methyl, tert-butylamino methyl, n-pentylamino methyl, isopentylamino methyl, neopentylamino methyl, tert-pentylamino methyl, n-hexylamino methyl, isohexylamino methyl, methylamino ethyl, methylamino propyl, methylamino isopropyl, methylamino butyl, methylamino isobutyl, methylamino pentyl, methylamino isopentyl, methylamino hexyl, methylamino isohexyl and the like.

The term “dialkylamino-C₁₋₆alkyl” in the present context designates a group (C₁₋₆alkyl)₂N—C₁₋₆-alkyl wherein C₁₋₆-alkyl is as defined above. Representative examples include, but are not limited to, dimethylamino methyl, diethylamino methyl (i.e. —CH₂—N—(C₂H₅)₂), dipropylamino methyl, di-isopropylamino methyl, dibutylamino methyl, di-isobutylamino methyl, di-sec-butylamino methyl, di-tert-butylamino methyl, dipentylamino methyl, di-isopentylamino methyl, di-neopentylamino methyl, di-tert-pentylamino methyl, dihexylamino methyl, diisohexylamino methyl, dimethylamino ethyl, dimethylamino propyl, dimethylamino isopropyl, dimethylamino butyl, dimethylamino isobutyl, dimethylamino pentyl, dimethylamino isopentyl, dimethylamino hexyl, dimethylamino isohexyl and the like.

“Halogen” designates an atom selected from the group consisting of F, Cl, Br and I.

The terms “unsubstituted” or “substituted” as used herein means that the groups in question are optionally unsubstituted or substituted with one, two or three substituents independently of each other selected from halogen, hydroxy, amino, mercapto, nitro, cyano, trifluoromethyl, trifluoromethylthio, trifluoromethoxy, C₁₋₆alkyl, C₁₋₆alkoxy, thioC₁₋₆alkyl, C₁₋₆alkylamino, alkylamino-C₁₋₆alkyl and dialkylamino-C₁₋₆alkyl. When the groups in question are substituted with more than one substituent the substituents may be the same or different.

Certain of the above defined terms may occur more than once in the structural formulae, and upon such occurrence each term shall be defined independently of the other.

As used herein, the phrase “a functional group which can be converted to hydrogen in vivo” is intended to include any group which upon administering the present compounds to the subjects in need thereof can be converted to hydrogen eg enzymatically or by the acidic environment in the stomach. Non-limiting examples of such groups are acyl, carbamoyl, monoalkylated carbamoyl, dialkylated carbamoyl, alkoxycarbonyl, alkoxyalkyl groups and the like such as C₁₋₆alkylcarbonyl, aroyl, C₁₋₆alkylcarbamoyl, di-C₁₋₆ alkyl-alkylcarbarnoyl, C₁₋₆ alkoxycarbonyl and C₁₋₆-alkoxy-C₁₋₆-alkyl.

As used herein, the phrase “diseases and disorders related to peptide deformylase” is intended to include any disease or disorder in which an effect, preferably an inhibiting effect, on peptide deformylase is beneficial, especially on the bacterial peptide deformylase.

The term “IC₅₀” as used herein denotes the concentration required for 50% inhibition of PDF in a binding assay.

Abbreviations and symbols commonly used in the peptide and chemical arts are used herein to describe the compounds of the present invention. In general, the amino acid abbreviations follow the IUPAC-IUB Joint Commission on Biochemical Nomenclature as described in Eur. J. Biochem., 158, 9 (1984).

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical.

### The Compounds

The present invention relates to compounds of the general formula (I)

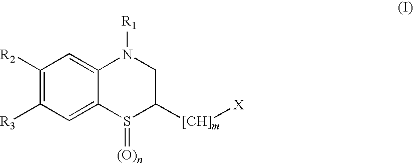

or a pharmaceutically acceptable salt or ester thereof, wherein

### X is —CONHOH, —COOH or —N(OH)CHO;

n is 0 (zero) or an integer 1 or 2;
m is an integer 1, 2, 3 or 4;
R₁ is selected from the group consisting of hydrogen, C₁₋₆ alkyl, C₂₋₆ alkenyl, C₃₋₁₀ cycloalkyl, C₁₋₆ alkyl-C₃₋₁₀ cycloalkyl, C₃₋₇ heterocycloalkyl, C₁₋₆ alkoxy, C₁₋₆ alkylamino, C₁₋₆ alkylmercapto, C₁₋₆ alkylhydroxy, thioC₁₋₆ alkyl, alkylamino-C₁₋₆alkyl, dialkylamino-C₁₋₆alkyl, an unsubstituted or substituted aryl group, an unsubstituted or substituted heteroaryl group, an unsubstituted or substituted C₁₋₆ alkylaryl group, and an unsubstituted or substituted C₁₋₆ alkylheteroaryl group; wherein a substituted group is substituted with one, two or three substituents independently selected from halogen, hydroxy, amino, mercapto, nitro, cyano, trifluoromethyl, C₁₋₆ alkyl, C₁₋₆ alkoxy and thioC₁₋₆ alkyl;
one of R₂ and R₃ is selected from the group consisting of halogen, hydrogen, carboxylic acid, —CONR₄R₅ and —CONHR₅, in which R₄ and R₅ are identical or different and independently of each other are selected from the group consisting of C₃₋₇ heterocycloalkyl, an unsubstituted or substituted aryl group, an unsubstituted or substituted heteroaryl group, an unsubstituted or substituted C₁₋₆ alkylaryl group, and an unsubstituted or substituted C₁₋₆ alkylheteroaryl group and an unsubstituted or substituted C₁₋₆ alkyl-C₃₋₇ heterocycloalkyl group; wherein a substituted group is substituted with one, two or three substituents independently selected from halogen, hydroxy, amino, mercapto, nitro, cyano, trifluoromethyl, C₁₋₆ alkyl, C₁₋₆ alkoxy, thioC₁₋₆alkyl, C₁₋₆alkylhydroxy, C₁₋₆alkylamino, alkylamino-C₁₋₆alkyl and dialkylamino-C₁₋₆alkyl; and
the other of R₂ and R₃ is selected from the group consisting of hydrogen, C₁₋₆ alkyl, C₂₋₆ alkenyl, C₃₋₁₀ cycloalkyl, C₁₋₆ alkyl-C₃₋₁₀ cycloalkyl, C₃₋₇ heterocycloalkyl, C₁₋₆ alkoxy, C₁₋₆ alkylamino, C₁₋₆ alkylmercapto, C₁₋₆ alkylhydroxy, thioC₁₋₆ alkyl, alkylamino-C₁₋₆alkyl, dialkylamino-C₁₋₆alkyl; an unsubstituted or substituted aryl group, an unsubstituted or substituted heteroaryl group, an unsubstituted or substituted C₁₋₆ alkylaryl group, and an unsubstituted or substituted C₁₋₆ alkylheteroaryl group; wherein a substituted group is substituted with one, two or three substituents independently selected from halogen, hydroxy, amino, mercapto, nitro, cyano, trifluoromethyl, C₁₋₆ alkyl, C₁₋₆ alkoxy, and thioC₁₋₆ alkyl.

In a preferred embodiment of the invention, X is —CONHOH. However, in other useful embodiments of the invention, X is —COOH or —N(OH)CHO.

In a preferred embodiment of the invention, n is 2. However, in other useful embodiments of the invention, n is 0 or n is 1, preferably 0.

In a preferred embodiment of the invention, m is 1. However, in other useful embodiments of the invention, m is 2, 3 or 4.

Preferably, R₁ is selected from the group consisting of hydrogen, C₁₋₆ alkyl, C₁₋₆ alkyl-C₃₋₁₀ cycloalkyl, C₁₋₆ alkylamino, C₁₋₆ alkylhydroxy, an unsubstituted or substituted C₁₋₆ alkylaryl group, and an unsubstituted or substituted C₁₋₆ alkylheteroaryl group; wherein a substituted group is substituted with one, two or three substituents independently selected from halogen, hydroxy, amino, mercapto, nitro, cyano, trifluoromethyl, C₁₋₆ alkyl, C₁₋₆ alkoxy, and thioC₁₋₆ alkyl. More preferably, R₁ is selected from hydrogen, methyl, ethyl, propyl, butyl, pentyl, methyl cyclopropyl, methyl cyclobutyl, methylcyclopentyl, methyl cyclohexyl, ethyl cyclohexyl, ethylamino, propylamino, butylamino, methylhydroxy, ethylhydroxy, propylhydroxy, butylhydroxy, benzyl, fluorosubstituted benzyl, chlorosubstituted benzyl, and bromo substituted benzyl; especially from hydrogen, ethyl, propyl, butyl, methylcyclopropyl, methylcyclobutyl, methylcyclopentyl, methylcyclohexyl, benzyl, and 3-fluorobenzyl.

In a preferred embodiment of the present invention, one of R₂/R₃ is selected among hydrogen, fluorine, chlorine, bromine, iodine and carboxylic acid.

In another preferred embodiment of the present invention, one of R₂/R₃ is —CONHR₅ or —CONR₄R₅:

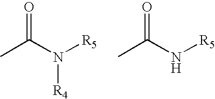

In yet another preferred embodiment of the present invention, one of R₂/R₃ is selected among C₃₋₇ heterocycloalkyl, an unsubstituted or substituted aryl group, an unsubstituted or substituted heteroaryl group, an unsubstituted or substituted C₁₋₆ alkylaryl group, and an unsubstituted or substituted C₁₋₆ alkylheteroaryl group; wherein a substituted group is substituted with one, two or three substituents independently selected from halogen, hydroxy, amino, mercapto, nitro, cyano, trifluoromethyl, C₁₋₆alkyl, C₁₋₆alkoxy and thioC₁₋₆ alkyl.

Most preferably, one of R₂/R₃ is hydrogen, C₃₋₇ heterocycloalkyl or C₃₋₇ heterocycloalkyl; especially hydrogen or 1-piperazinyl.

When one of R₂/R₃ is —CONHR₅ or —CONR₄R₅, R₄ or R₅ is independently of each other preferably C₃₋₇ heterocycloalkyl, C₁₋₆ alkyl-C₃₋₇ heterocycloalkyl, heteroaryl or C₁₋₆ alkylheteroaryl having one or more heteroatoms selected among N, O and S; or an unsubstituted or substituted aryl group, an unsubstituted or substituted heteroaryl group, an unsubstituted or substituted C₁₋₆ alkylaryl group, and an unsubstituted or substituted C₁₋₆ alkylheteroaryl group; wherein a substituted group is substituted with one, two or three substituents independently selected from halogen, hydroxy, amino, mercapto, nitro, cyano, trifluoromethyl, C₁₋₆ alkyl, C₁₋₆ alkoxy, C₁₋₆ alkylthio, C₁₋₆ alkylhydroxy, C₁₋₆ alkylamino, alkylamino-C₁₋₆ alkyl and dialkylamino-C₁₋₆alkyl.

More preferably, R₄ or R₅ is independently selected from a group consisting of benzyl; mono-, di-, or tri-fluoro-substituted benzyl, mono-, di-, or -tri-bromo-substituted benzyl, trifluoromethyl substituted benzyl, methoxy substituted benzyl, trifluoromethoxy substituted benzyl, dimethylamino substituted benzyl, nitro substituted benzyl, 5-thiophen-2-yl-2H-pyrazol-3-yl, 8-methyl-8-aza-bicyclo[3.2.1]oct-3-yl, methylpyridyl, methyl-2-thienyl, 3-pyrazolyl, 2-thiazolyl, 4-methyl-1-piperazinyl.

Preferred compounds of the invention are:
2-(3,4-Dihydro-2H-benzo[1,4]thiazin-2-yl)-N-hydroxy-acetamide
2-(1,1-Dioxo-1,2,3,4-tetrahydro-1λ⁶-benzo[1,4]thiazin-2-yl)-N-hydroxy-acetamide
2-(4-Ethyl-3,4-dihydro-2H-benzo[1,4]thiazin-2-yl)-N-hydroxy-acetamide
2-(4-Ethyl-1,1-dioxo-1,2,3,4-tetrahydro-1λ⁶-benzo[1,4]thiazin-2-yl)-N-hydroxy-acetamide
N-Hydroxy-2-(4-propyl-3,4-dihydro-2H-benzo[1,4]thiazin-2-yl)acetamide
2-(1,1-Dioxo-4-propyl-1,2,3,4-tetrahydro-1λ⁶-benzo[1,4]thiazin-2-yl)-N-hydroxy-acetamide
2-(4-Butyl-3,4-dihydro-2H-benzo[1,4]thiazin-2-yl)-N-hydroxy-acetamide
2-(4-Butyl-1,1-dioxo-1,2,3,4-tetrahydro-1λ⁶-benzo[1,4]thiazin-2-yl)-N-hydroxy-acetamide
2-(4-Benzyl-1,1-dioxo-1,2,3,4-tetrahydro-1λ⁶-benzo[1,4]thiazin-2-yl)-N-hydroxy-acetamide
2-[4-(3-Fluoro-benzyl)-1,1-dioxo-1,2,3,4-tetrahydro-1λ⁶-benzo[1,4]thiazin-2-yl)-N-hydroxy-acetamide
4-Ethyl-2-hydroxycarbamoylmethyl-1,1-dioxo-1,2,3,4-tetrahydro-1λ⁶-benzo[1,4]thiazine-6-carboxylic acid benzylamide
4-Ethyl-2-hydroxycarbamoylmethyl-1,1-dioxo-1,2,3,4-tetrahydro-1λ⁶-benzo[1,4]thiazine-6-carboxylic acid 2-fluoro-benzylamide
4-Ethyl-2-hydroxycarbamoylmethyl-1,1-dioxo-1,2,3,4-tetrahydro-1λ⁶-benzo[1,4]thiazine-6-carboxylic acid 3-fluoro-benzylamide
4-Ethyl-2-hydroxycarbamoylmethyl-1,1-dioxo-1,2,3,4-tetrahydro-1λ⁶-benzo[1,4]thiazine-6-carboxylic acid 4-fluoro-benzylamide
4-Ethyl-2-hydroxycarbamoylmethyl-1,1-dioxo-1,2,3,4-tetrahydro-1λ⁶-benzo[1,4]thiazine-6-carboxylic acid 2-bromo-benzylamide
4-Ethyl-2-hydroxycarbamoylmethyl-1,1-dioxo-1,2,3,4-tetrahydro-1λ⁶-benzo[1,4]thiazine-6-carboxylic acid 3-bromo-benzylamide
4-Ethyl-2-hydroxycarbamoylmethyl-1,1-dioxo-1,2,3,4-tetrahydro-1λ⁶-benzo[1,4]thiazine-6-carboxylic acid 4-bromo-benzylamide
4-Ethyl-2-hydroxycarbamoylmethyl-1,1-dioxo-1,2,3,4-tetrahydro-1λ⁶-benzo[1,4]thiazine-6-carboxylic acid 2-nitro-benzylamide
4-Ethyl-2-hydroxycarbamoylmethyl-1,1-dioxo-1,2,3,4-tetrahydro-1λ⁶-benzo[1,4]thiazine-6-carboxylic acid 3-nitro-benzylamide
4-Ethyl-2-hydroxycarbamoylmethyl-1,1-dioxo-1,2,3,4-tetrahydro-1λ⁶-benzo[1,4]thiazine-6-carboxylic acid 4-nitro-benzylamide
4-Ethyl-2-hydroxycarbamoylmethyl-1,1-dioxo-1,2,3,4-tetrahydro-1λ⁶-benzo[1,4]thiazine-6-carboxylic acid 2-methoxy-benzylamide
4-Ethyl-2-hydroxycarbamoylmethyl-1,1-dioxo-1,2,3,4-tetrahydro-1λ⁶-benzo[1,4]thiazine-6-carboxylic acid 3-methoxy-benzylamide
4-Ethyl-2-hydroxycarbamoylmethyl-1,1-dioxo-1,2,3,4-tetrahydro-1λ⁶-benzo[1,4]thiazine-6-carboxylic acid 4-methoxy-benzylamide
4-Ethyl-2-hydroxycarbamoylmethyl-1,1-dioxo-1,2,3,4-tetrahydro-1λ⁶-benzo[1,4]thiazine-6-carboxylic acid 3-trifluoromethyl-benzylamide
4-Ethyl-2-hydroxycarbamoylmethyl-1,1-dioxo-1,2,3,4-tetrahydro-1λ⁶-benzo[1,4]thiazine-6-carboxylic acid 4-trifluoromethyl-benzylamide
4-Ethyl-2-hydroxycarbamoylmethyl-1,1-dioxo-1,2,3,4-tetrahydro-1λ⁶-benzo[1,4]thiazine-6-carboxylic acid 4-trifluoromethoxybenzylamide
4-Ethyl-2-hydroxycarbamoylmethyl-1,1-dioxo-1,2,3,4-tetrahydro-1λ⁶-benzo[1,4]thiazine-6-carboxylic acid 4-dimethylaminobenzylamide
4-Ethyl-2-hydroxycarbamoylmethyl-1,1-dioxo-1,2,3,4-tetrahydro-1λ⁶-benzo[1,4]thiazine-6-carboxylic acid (pyridin-4-ylmethyl)-amide
4-Ethyl-2-hydroxycarbamoylmethyl-1,1-dioxo-1,2,3,4-tetrahydro-1λ⁶-benzo[1,4]thiazine-6-carboxylic acid (thiophen-2-ylmethyl)amide
4-Ethyl-2-hydroxycarbamoylmethyl-1,1-dioxo-1,2,3,4-tetrahydro-1λ⁶-benzo[1,4]thiazine-6-carboxylic acid (1H-pyrazol-3-yl)-amide
4-Ethyl-2-hydroxycarbamoylmethyl-1,1-dioxo-1,2,3,4-tetrahydro-1λ⁶-benzo[1,4]thiazine-6-carboxylic acid thiazol-2-ylamide
2-[4-Ethyl-6-(4-methyl-piperazine-1-carbonyl)-1,1-dioxo-1,2,3,4-tetrahydro-1λ⁶-benzo[1,4]thiazin-2-yl]-N-hydroxy-acetamide
4-Ethyl-2-hydroxycarbamoylmethyl-1,1-dioxo-1,2,3,4-tetrahydro-1λ⁶-benzo[1,4]thiazine-6-carboxylic acid (5-thiophen-2-yl-2H-pyrazol-3-yl)-amide
4-Ethyl-2-hydroxycarbamoylmethyl-1,1-dioxo-1,2,3,4-tetrahydro-1λ⁶-benzo[1,4]thiazine-6-carboxylic acid (8-methyl-8-aza-bicyclo[3.2.1]oct-3-yl)-amide
2-(4-Cyclopropylmethyl-1,1-dioxo-1,2,3,4-tetrahydro-1λ⁶-benzo[1,4]thiazin-2-yl)-N-hydroxy-acetamide
2-(4-Cyclobutylmethyl-1,1-dioxo-1,2,3,4-tetrahydro-1λ⁶-benzo[1,4]thiazin-2-yl)-N-hydroxy-acetamide
2-(4-Cyclopentylmethyl-1,1-dioxo-1,2,3,4-tetrahydro-1λ⁶-benzo[1,4]thiazin-2-yl)-N-hydroxy-acetamide, and
2-(4-Cyclohexylmethyl-1,1-dioxo-1,2,3,4-tetrahydro-1λ⁶-benzo[1,4]thiazin-2-yl)-N-hydroxy-acetamide.

The compounds of the invention may exist as geometric isomers or optical isomers or stereoisomers as well as tautomers. Accordingly, the invention includes all geometric isomers and tautomers including mixtures and racemic mixtures of these and a pharmaceutically acceptable salt thereof, especially all R- and S-isomers. The compounds of the invention may also exist as solvent complexes as well as in different morphological forms.

The present invention also encompasses pharmaceutically acceptable salts of the present compounds. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic adds include hydrochloric, hydrobromic, hydroiodic, phosphoric, sulfuric, nitric adds and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

Also intended as pharmaceutically acceptable acid addition salts are the hydrates and solvent complexes, which the present compounds are able to form.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of the present invention may form solvates with standard low molecular weight solvents using methods well known to the person skilled in the art. Such solvates are also contemplated as being within the scope of the present invention.

The invention also encompasses prodrugs of the present compounds, which on administration undergo chemical conversion by metabolic processes before becoming active pharmacological substances. In general, such prodrugs will be functional derivatives of the present compounds, which are readily convertible in vivo into the required compound of the Formula I. Prodrugs are any covalently bonded compounds, which release the active parent drug according to Formula I in vivo. If a chiral center or another form of an isomeric center is present in a compound of the present invention, all forms of such isomer or isomers, including enantiomers and diastereomers, are intended to be covered herein. Inventive compounds containing a chiral center may be used as a racemic mixture, an enantiomerically enriched mixture, or the racemic mixture may be separated using well-known techniques and an individual enantiomer may be used alone. In cases wherein compounds may exist in tautomeric forms, such as keto-enol tautomers, each tautomeric form is contemplated as being included within this invention whether existing in equilibrium or predominantly in one form. Conventional procedures for the selection and preparation of suitable prodrug derivatives are described, for example, in “Design of Prodrugs”, ed. H. Bundgaard, Elsevier, 1985.

The invention also encompasses active metabolites of the present compounds.

The present invention includes all complexes of the compounds of this invention.

The meaning of any substituent at any one occurrence in Formula I or any subformula thereof is independent of its meaning, or any other substituent's meaning, at any other occurrence, unless specified otherwise.

In a preferred embodiment of this invention, the compounds of Formula I exhibit an IC₅₀ value of less than 500 μM, preferably less than 100 μM, more preferably less than 50 μM, even more preferably less than 1 μM, especially less than 500 nM, particularly less than 100 nM, when subjected to a bacterial PDF assay.

### Synthetic Method of Preparation

The compounds of the present invention having the general Formula I may be prepared by the general methods set forth in the scheme A, B, C, D, E and F below, further details of the synthesis are described in the “materials and methods” section.

Compounds, wherein R₁ is C₁₋₆ alkyl-R, X is —CONHOH, and m is 1, can be synthesized as depicted in scheme A. The amino thiol is first acylated and consecutive Michael addition n (step 1) to yield intermediate (I). Intermediate (I) is then esterified in step 2 to give intermediate (II). The intermediate (II) is reduced (step 3) using borane. Acylation of (III) in step 4 using the appropriate acid chlorides gives intermediates (IV). Oxidation of intermediates (IV) using sodium perborate (to give intermediate (V). The reaction time was 14-16 h. Reduction of the reaction time to a couple of hours resulted in mono-oxidation of the sulphur and hence title compounds having n=1 as a result), reduction (in step 6) using borane (to give intermediates (VI)) and finally hydrolysis using hydroxylamine (step 7) gave the desired products (VII).

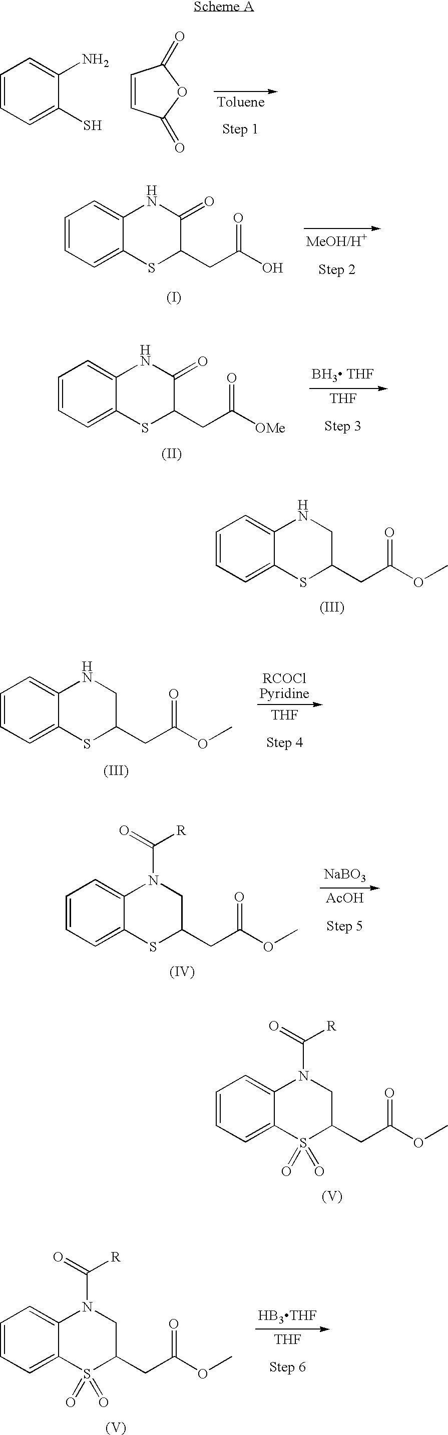

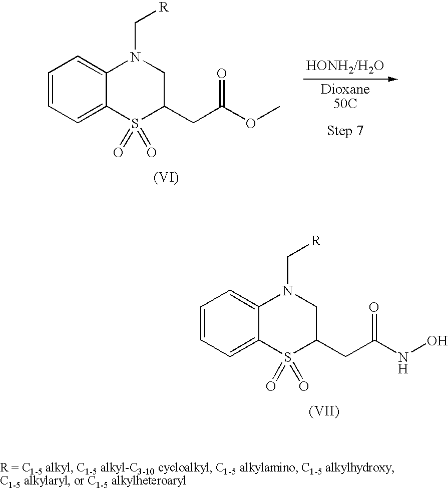

Compounds, wherein R₁ is hydrogen, X is —CONHOH, and m is 1, can be synthesized as depicted in scheme B. Intermediate (III) was synthesized as in method A and was further oxidized with NaBO₃ in acetic acid in step 1 to give intermediate (IV). Hydrolysis of the ester functionality with hydroxylamine in a mixture of water and dioxane (step 2) ultimately yielded the desired product (V).

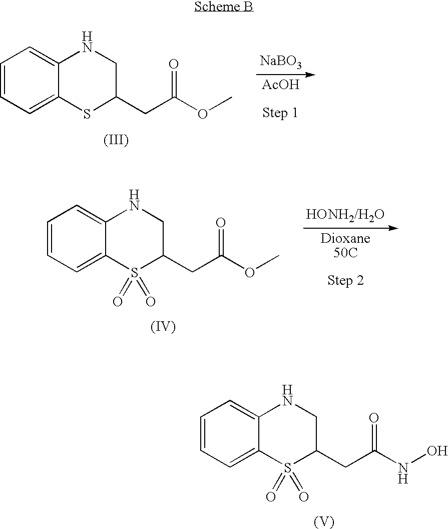

Compounds, wherein R₁ is C₁₋₆alkyl-R, X is —CONHOH, m is 1 and n is 0, can be synthesized as depicted in scheme C. Intermediate (III) was synthesized as in method A and was further reduced using borane in tetrahydrofuran in step 1 to give intermediate (IV), which as in methods A and B upon hydrolysis with hydroxylamine yielded the desired products (V).

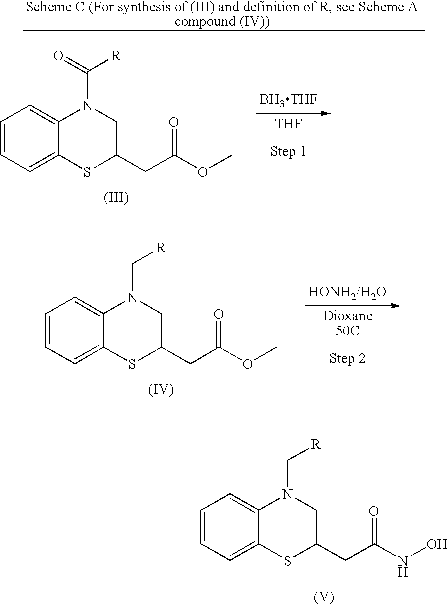

Compounds, wherein R₁ is Con alkyl, X is CONHOH, m is 1 and n is 2, can be synthesized as depicted in scheme D. The acid chloride was esterified using fluorenyl methanol (step 1) to give intermediate (I). Nucleophilic aromatic addition of (II) to (I) in step 2 using Hünig's base resulted in intermediate (III). Reduction of the amino functionality (step 3) and subsequent ring closure of intermediate (III) gave (IV). Reduction in step 4 using borane and thereafter acetylation in step 5 resulted in intermediate (VI). Oxidation (step 6) using sodium perborate and reduction (step 7) using borane gave intermediate (VIII). Deprotection of the acid functionality (step 8) using diethylamine gave intermediate (X), which was used to create the amide library in the following reaction steps using the appropriate amine, TBTU and NEM (step 9). Hydrolysis using hydroxylamine in step 10 gave the desired amides (XI) of scheme D.

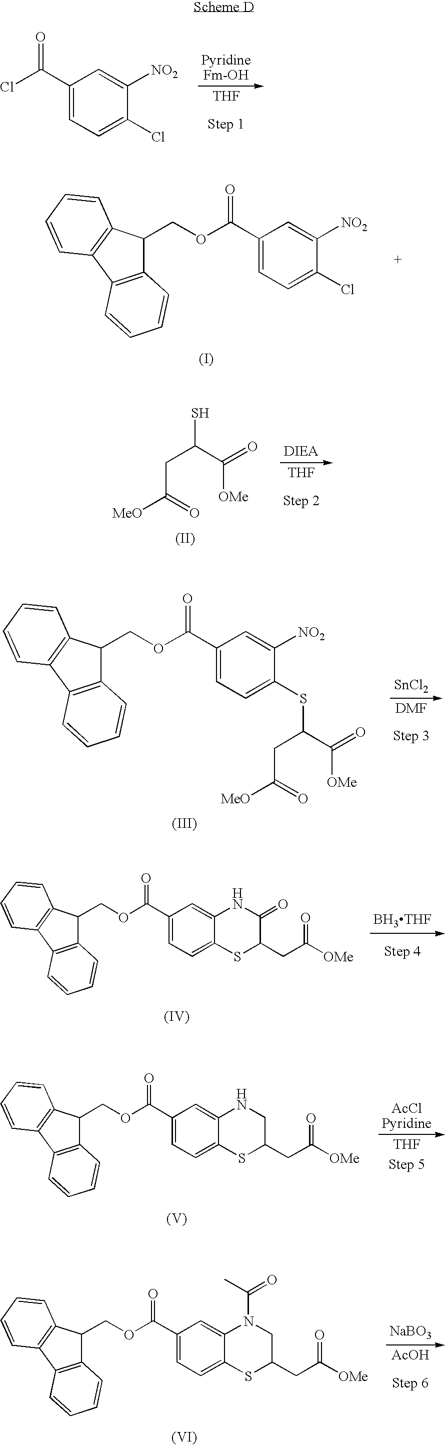

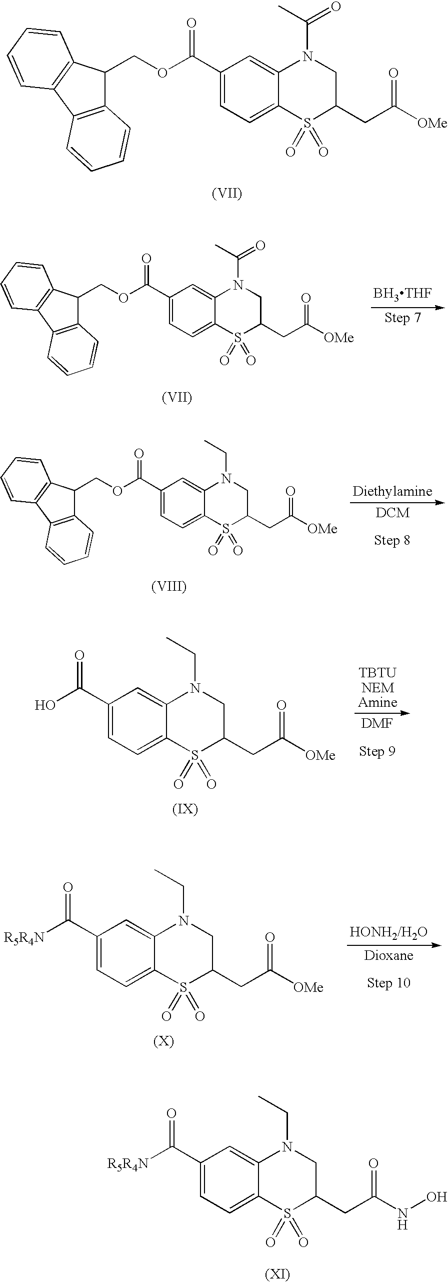

The corresponding carboxylic acids, wherein X is —COOH, can be synthesized as depicted in scheme E, by treatment of intermediates (VI), (IV), (IV) and (X) in schemes A, B, C and D respectively with sodium hydroxide and thereafter acid.

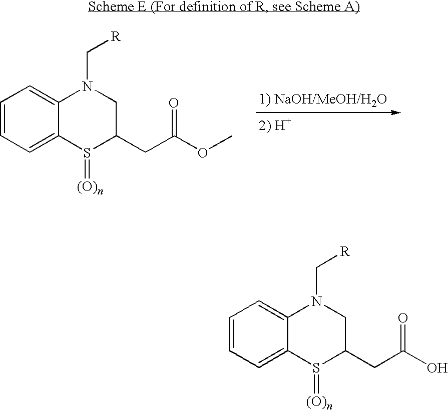

It can be contemplated that the synthesis of the corresponding analogues, wherein X is —C(OH)CHO, can be synthesized as depicted in scheme F. The starting alcohol (I) can be protected with TBDMS-Cl and thereafter the amide can be reduced to the corresponding amine by borane as in step 4, scheme D. The formed amine can then be acylated with an acid chloride (RCOCl) in pyridine, to yield a second amide product. Oxidation of the sulfur can be performed by MCPBA. A final reduction of the second amide can then be performed by borane as in step 7, scheme D. Deprotection of the alcohol by TBAF and a functional group transformation by PBr₃, will form the corresponding bromide. Reaction of the bromide and H₂NO^{t}Bu will yield the protected hydroxylamine which is formylated by CDI/HCOOH. A final treatment with acid will give the desired compounds, wherein X is —C(OH)CHO. This method can easily be adapted to all oxidation states of the thio-moiety, i.e. n can be 0, 1 or 2.

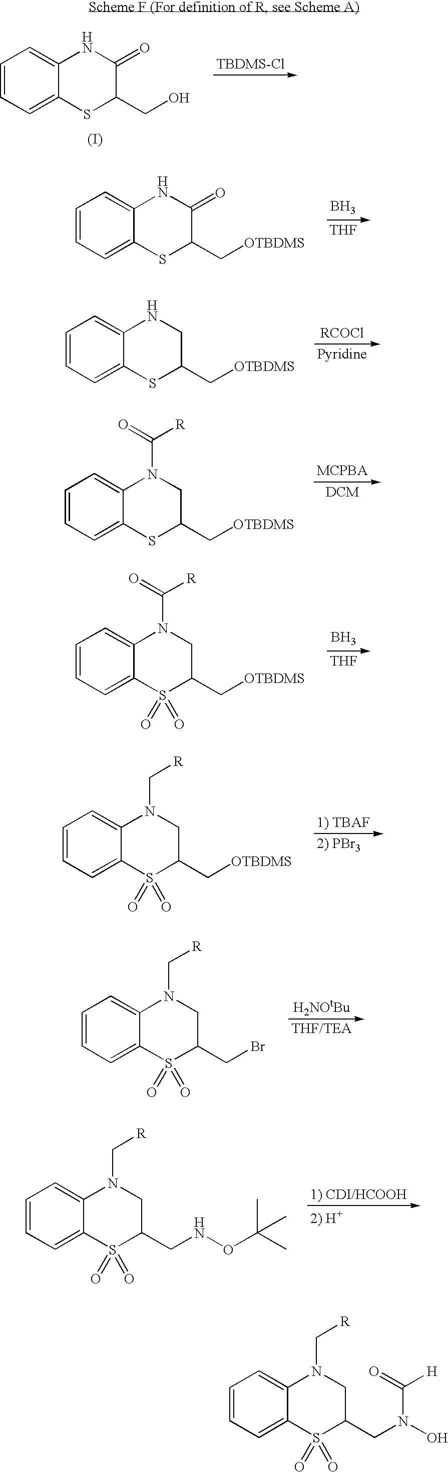

Acid addition salts of the compounds of Formula I are prepared in a standard manner in a suitable solvent from the parent compound and an excess of an acid, such as hydrochloric, hydrobromic, hydrofluoric, sulfuric, phosphoric, acetic, trifluoroacetic, maleic, succinic or methanesulfonic. Certain of the compounds form Inner salts or zwitterions, which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts. Halides, sulfate, phosphate, alkanoates (such as acetate and trifluoroacetate), benzoates, and sulfonates (such as mesylate) are examples of anions present in pharmaceutically acceptable salts.

### Pharmaceutical Compositions

In one aspect of this invention, there is provided a pharmaceutical composition comprising, as an active ingredient, a compound of the present invention together with a pharmaceutically acceptable carrier or diluent. This composition may be in unit dosage form and may comprise from about 1 μg to about 1000 mg such as, e.g., from about 10 μg to about 500 mg, preferably from about 0.05 to about 100 mg or more preferably from about 0.1 to about 50 mg, of the compound of the invention or a pharmaceutically acceptable salt or ester thereof. The composition of the invention may be used for oral, nasal, transdermal, pulmonal or parenteral administration. It is contemplated that the pharmaceutical composition of the invention is useful for treatment of bacterial and/or parasitic infections.

The compounds of the invention may be administered alone or in combination with pharmaceutically acceptable carriers, diluents or excipients, in either single or multiple doses. Accordingly, the compounds of Formula I may be used in the manufacture of a medicament. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19.sup.th Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

A typical oral dosage is in the range of from about 0.001 to about 50 mg/kg body weight per day, preferably from about 0.01 to about 30 mg/kg body weight per day, and more preferred from about 0.05 to about 20 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from 1 μg to about 1000 mg such as, e.g., from about 10 μg to about 500 mg, preferably from about 0.05 to about 100 mg, more preferably from about 0.1 to about 50 mg, and more preferred from about 0.5 mg to about 20 mg.

For parenteral routes, such as intravenous, intrathecal, intramuscular and similar administration, typically doses are in the order of about half the dose employed for oral administration.

The compounds of this invention are generally utilized as the free substance or as a pharmaceutically acceptable salt thereof. One example is an acid addition salt of a compound having the utility of a free base. When a compound of the Formula (I) contains a free base such salts are prepared in a conventional manner by treating a solution or suspension of a free base of the Formula (I) with a chemical equivalent of a pharmaceutically acceptable acid, for example, inorganic and organic acids. Representative examples are mentioned above. Physiologically acceptable salts of a compound with a hydroxy group include the anion of said compound in combination with a suitable cation such as sodium or ammonium ion.

For parenteral administration, solutions of the novel compounds of the Formula (I) in sterile aqueous solution, aqueous propylene glycol or sesame or peanut oil may be employed. Such aqueous solutions should be suitable buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water. Similarly, the carrier or diluent may include any sustained release material known in the art, such as glyceryl monostearate or glyceryl distearate, alone or mixed with a wax. The pharmaceutical compositions formed by combining the novel compounds of the Formula (I) and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active Ingredient, and which may include a suitable excipient. These formulations may be in the form of powder or granules, as a solution or suspension in an aqueous or non-aqueous liquid, or as an oil-in-water or water-in-oil liquid emulsion.

If a solid carrier is used for oral administration, the preparation may be tableted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet, which may be prepared by conventional tabletting techniques, may contain:

Core:

| | |
|---|---|
| Active compound (free compound or salt) | 5.0 mg |
| Lactosum Ph. Eur. | 67.8 mg |
| Cellulose, microcryst. (Avicel) | 31.4 mg |
| Amberlite | 1.0 mg |
| Magnesii stearas | q.s. |

Coating:

| | |
|---|---|
| Hydroxypropyl methylcellulose approx. | 9 mg |
| Acylated monoglyceride approx. | 0.9 mg |

If desired, the pharmaceutical composition of the invention may comprise the compound of the Formula (I) in combination with further pharmacologically active substances such as those described in the foregoing.

### Use of the Invention

The compounds of Formula I are useful as protease inhibitors, particularly as inhibitors of metallo proteases, more particularly as inhibitors of peptide deformylase, even more particularly as inhibitors of bacterial peptide deformylase. The present invention provides useful compositions and formulations of said compounds, including pharmaceutical compositions and formulations of said compounds.

The compounds of the present invention may be especially useful for the treatment or prevention of diseases caused by a variety of bacterial or prokaryotic organisms. Examples include Gram-positive and Gram-negative aerobic and anaerobic bacteria such as, Staphylococci, for example S. aureus and S. epidermidis; Enterococci, for example E. faecium and E. faecalis; Streptococci, for example S. pneumoniae; Haemophilus, for example H. influenzae; Moraxella, for example M. catarrhalis; Escherichia, for example E. coli; Mycobacteria, for example M. tuberculosis and M. ranae; Mycoplasma, for example M. pneumoniae; Pseudomonas, for example P. aeruginosa; intercellular microbes, for example Chlamydia and Rickettsiae. Other examples include Klebsiella pneumoniae, Shigella flexneri, Salmonella typhimurium, Bordetella pertussis, Clostridia perfringens, Helicobacter pylori, Campylobacter jejuni, Legionella pneumophila and Neisseria gonorrhoeae. It is further contemplated that the compounds of the present invention are useful for the treatment of parasitic infections, for example infections caused by Plasmodium falciparum and the like.

Accordingly, in one aspect the present invention relates to a method for the treatment of ailments, the method comprising administering to a subject in need thereof an effective amount of a compound or a composition of this invention. It is contemplated that an effective amount of a compound or a composition of this invention corresponds to an amount of active ingredient, i.e. active compound or a pharmaceutically acceptable salt or ester thereof, in the range of from about 1 μg to about 1000 mg such as, e.g., from about 10 μg to about 500 mg, preferably from about 0.05 to about 100 mg or more preferably from about 0.1 to about 50 mg per day.

In yet another aspect, the present invention relates to use of a compound of this invention for the preparation of a medicament, preferably a medicament for the treatment of infections caused by Gram-positive or Gram-negative aerobic or anaerobic bacteria, or by parasites.

In a preferred embodiment of the invention, there is provided a medicament for the treatment of infections caused by Staphylococci, Enterococci, Streptococci, Haemophilus, Moraxella, Escherichia, Mycobacteria, Mycoplasma, Pseudomonas, Chlamydia, Rickettsia, Klebsiella, Shigella, Salmonella, Bordetella, Clostridia, Helicobacter, Campylobacter, Legionella and Neisseria, preferably caused by Staphylococcus aureus, Staphylococcus epidermidis, Enterococcus faecium, Enterococcus faecalis, Streptococcus pneumoniae, Haemophilus influenzae, Moraxella catarrhalis, Escherichia coli, Mycobacterium tuberculosis, Mycobacterium ranae, Mycoplasma pneumoniae, Pseudomonas aeruginosa, Chlamydia, Rickettsiae, Klebsiella pneumoniae, Shigella flexneri, Salmonella typhimurium, Bordetella pertussis, Clostridia perfringens, Helicobacter pylori, Campylobacter jejuni, Legionella pneumophila and Neisseria gonorrhoeae.

It is further contemplated that the compounds of the present invention are useful for the treatment of parasitic infections, for example infections caused by Plasmodium falciparum and the like.

An intravenous infusion of the compound in 5% dextrose in water or normal saline, or a similar formulation with suitable excipients, is most effective, although an intramuscular bone injection is also useful. Typically, the parenteral dose will be about 0.01 to about 100 mg/kg; preferably between 0.1 and 20 mg/kg, in a manner to maintain the concentration of drug in the plasma at a concentration effective to inhibit PDF. The compounds may be administered one to four times daily at a level to achieve a total daily dose of about 0.4 to about 400 mg/kg/day. The precise amount of an inventive compound which is therapeutically effective, and the route by which such compound Is best administered, is readily determined by one of ordinary skill in the art by comparing the blood level of the agent to the concentration required to have a therapeutic effect.

The compounds of this invention may also be administered orally to the patient, in a manner such that the concentration of drug is sufficient to inhibit bone resorption or to achieve any other therapeutic indication as disclosed herein. Typically, a pharmaceutical composition containing the compound is administered at an oral dose of between about 0.1 to about 50 mg/kg in a manner consistent with the condition of the patient. Preferably the oral dose would be about 0.5 to about 20 mg/kg.

No unacceptable toxicological effects are expected when compounds of the present invention are administered in accordance with the present invention.

The compounds of the present invention fully or partly inhibit bacterial PDF, and are thus useful for the treatment and/or prevention of a wide variety of conditions and disorders in which inhibition of PDF is beneficial.

Accordingly, in another aspect the present invention relates to a compound of the general Formula (I) or any optical or geometric isomer or tautomeric form thereof including mixtures of these or a pharmaceutically acceptable salt thereof for use as a pharmaceutical composition.

The invention also relates to pharmaceutical compositions comprising, as an active ingredient, at least one compound of the Formula (I) or any optical or geometric isomer or tautomeric form thereof including mixtures of these or a pharmaceutically acceptable salt thereof together with one or more pharmaceutically acceptable carriers or diluents.

In the following synthetic examples, all of the starting materials were obtained from commercial sources unless otherwise indicated. Without further elaboration, it is believed that one skilled in the art can, using the preceding description, utilize the present invention to its fullest extent. These examples are given to illustrate the invention, not to limit its scope.

### EXAMPLES

### Materials and Methods

The starting materials used herein are commercially available or can be prepared according to procedures previously reported in the literature. Unless otherwise stated commercial starting materials were used without further purification. All solvents were HPLC grade. Anhydrous solvents were obtained by storing over 4 Å activated molecular sieves. Synthetic methods to prepare the compounds of this invention might employ protective groups to mask a reactive functionality or minimize unwanted side reactions. Such protective groups are described generally in Green (1981).

Room temperature is approx. 20° C. Mass spectra (ES-MS spectra) were obtained on a Micromass Quattro micro instrument in the positive mode unless otherwise noted.

### Materials and Abbreviations

AcOH Acetic acid
CDI 1,1′-Carbonyldiimidazole
DCM Dichloromethane
DIEA Diisopropylethyl amine
DMF N,N-Dimethyl formamide
Fm 9-Fluorenylmethyl
Fmoc 9-Fluorenylmethoxycarbonyl
MCPBA m-Chloroperbenzoic acid
NEM N-Ethyl morpholine (from Fluka; 98%)
TBAF Tetrabutylammonium fluoride
TBDMS tert-Butyldimethylsilyl
TBTU O-Benzotriazol-1-yl-N,N,N′,N′-tetramethyluronium tetrafluoroborate (from Fluka; 98%)
TFA Trifluoroacetic acid
THF Tetrahydrofurane
TNBF 2,4,6-trinitrobenzene sulfonic acid

Piperidine was obtained from Fluka (98%). 1,2,4-Trifluoro-5-nitrobenzene was obtained from Aldrich (99%). 4-Chloro-3-nitrobenzoyl chloride obtained from Aldrich (98%). SnCl₂ was obtained from Fluka (98%). Maleic anhydride was obtained from Aldrich (95%). Thiomalic acid was obtained from Aldrich (97%). 9-Fluorenemethanol was obtained from Fluka (98%). Borane in THF (1M solution) was purchased from Aldrich. Hydroxylamine in water (50% solution) was purchased from Fluka. Sodium perborate tetrahydrate (97%) was purchased from Fluka. 1-Fmoc-piperazine hydrochloride was obtained from NeoSys (99%). 2-Aminothiophenol was obtained from Aldrich (99%). All used amines were obtained from Aldrich (purity 95-98%).

### Synthesis of Compounds of the Invention

Illustrative general methods for the synthesis of the compounds of the invention are described hereinbefore and illustrated in Schemes A, B, C, D, E and F respectively. Detailed descriptions of method A to D are described below.

### Method A (Scheme A)

### Step 1:

2-Aminothiophenol (120 mmol, 12.81 ml) was dissolved in toluene. Maleic anhydride (129 mmol, 12.6 g, 1.075 equiv.) was added to the solution. A precipitate was formed shortly after completion of addition of the maleic an hydride. The reaction was allowed to continue for another 3 hours at room temperature. The precipitate was collected and washed with several portions of toluene. Yield: 92%

### Step 2:

The product from step 1 (I) (110 mmol, 24.5 g), was dissolved in methanol (acidified by addition of a few drops of acetyl chloride). The resulting solution was stirred at room temperature over night. The solvent was removed in vacuo to give (II) in scheme A. Yield: Quantitative.

### Step 3:

The product from step 2 (II) (110 mmol, 26.5 g), was dissolved in dry THF, and cooled on an Ice-bath under inert atmosphere (Ar). Borane in THF (132 mmol, 1M, 1.2 equiv) was added. After completion of addition of borane the ice-bath was removed and the reaction mixture was stirred over night at room temperature. The solvent was removed in vacuo. Water was added along with EtOAc. The organic phase was separated and the aqueous phase was extracted twice with EtOAc. The organic layers were combined and dried over Na₂SO₄. Removal of the organic phase yielded a yellow oil. The crude product was passed through a silica column using 50% EtOAc in heptane as eluent. Yield: 66%

### Step 4:

The appropriate acyl chloride (1.1 equiv, typically 3.3 mmol) was dissolved in dry THF, and pyridine was added. A precipitate formed and a solution (in THF) of the product from step 3 (III) (typically 3.0 mmol, 0.67 g) was added. The resulting mixture was stirred at room temperature over night. The solvent was removed in vacuo. EtOAc was added and the resulting solution was washed with 1M HCl and there after with sat. NaHCO₃. The organic phase was dried over Na₂SO₄ and the solvent removed in vacuo. The product was used in the next step without further purification.

### Step 5:

The products formed in step 4 (IV) (typically 1.1-1.6 mmol) were dissolved in AcOH and NaBO₃ (4-5 equiv.) was added. The resulting solutions were heated to 50° C. over night. The solvent was removed in vacuo, and the residues were partitioned between EtOAc and water. The organic phase was separated and dried over Na₂SO₄. The solvent was removed and the products were used without further purification.

### Step 6:

The products from step 5 (V) were dissolved in dry THF and BH₃ in THF (1M, 1.2 equiv.) was added and the resulting solution was stirred at room temperature over night. The solvent was removed and sat. NaHCO₃ along with EtOAc was added. The organic phase was collected and dried over Na₂SO₄. The crude products were purified using preparative HPLC.

### Step 7:

The products from step 6 (VI) (typically 0.4-0.1 mmol) were dissolved in dioxane and hydroxylamine (50% in water, typically 8-10 equiv.) was added. The resulting solution was heated to 50° C. over night. The solutions were acidified (pH=2) with TFA and purified by preparative HPLC to give the desired products (VIII).

### Method B (Scheme B)

### Step 1:

Intermediate (III) was synthesized in an identical manner to method A. Intermediate (III) (0.45 mmol, 100 mg) was dissolved in AcOH and NaBO₃ (5 equiv., 2.24 mmol, 345 mg). The procedure was then performed identical to step 5 in method A.

### Step 2:

Intermediate (IV) was treated in an identical manner to method A, step 7 to yield (V).

### Method C (Scheme C)

### Step 1:

Performed in an identical manner to method A, step 6.

### Step 2:

Performed in an identical manner to method A, step 7.

### Method D (Scheme D)

### Step 1:

4-Chloro-3-nitrobenzoyl chloride (1.13 equiv., 133.2 mmol, 29.3 g) was dissolved in dry THF and pyridine (2 equiv., 235.8 mmol, 19.0 ml) was added. A white precipitate formed immediately upon addition of pyridine. A solution of 9-fluorenemethanol (1 equiv., 117.9 mmol, 23.1 g) in dry THF was added and the resulting mixture was stirred at room temperature over night. The solvent was removed in vacuo and the resulting solid was mixed with diethyl ether and filtered. The solid was then washed several times with ether to achieve the pure desired product. Yield: 95%.

### Step 2:

The intermediate from step 1 (I) (1 equiv., 60 mmol, 22.8 g) was dissolved in THF and a solution of dimethylthiomaleate (II) (1 equiv. 60 mmol, 10.4 g) in THF was added. To the resulting solution was added DIEA (1.2 equiv., 72 mmol, 12.3 ml). Upon addition of the base the solution turned into a deep orange colour. Stirring was continued at room temperature over night. The solvent was removed and the resulting crude product was purified by column chromatography using 50% EtOAc in heptane as eluent. The pure product was retrieved as an orange oil. Yield: 88%

### Step 3:

The product from step 2 (III) (1 equiv., 52.8 mmol, 27.5 g) was mixed with a solution of SnCl₂ (200 ml of a 2M solution). The resulting solution was stirred at room temperature over night. The solution had turned pale yellow after reduction of the nitro group. The solvent was removed in vacuo, and the crude product was passed through a short silica column using 50% EtOAc in chloroform as eluent. Removing the solvent produced an oil which was re-dissolved in EtOAc and washed with water. The organic phase was removed and dried over Na₂SO₄. Yield: Quantitative.

### Step 4:

The product from step 3 (IV) (1 equiv., 60 mmol, 26.3 g) was dissolved in dry THF and BH₃ in THF (1M, 1.2 equiv, 72 mmol, 72 ml) was added. During addition of BH₃ the reaction solution was cooled on an ice-bath. The reaction solution was stirred at room temperature over night. The solvent was removed in vacuo and water and EtOAc was added to the solid residue. The organic phase was collected and the aqueous phase was extracted with EtOAc. The collected organic layers were combined and washed with brine. Drying over Na₂SO₄ yielded a clear oil. Yield: 50%

### Step 5:

The product from step 4 (V) (1 equiv., 11 mmol, 4.71 g) was dissolved in THF and acetyl chloride (3 equiv., 33 mmol, 2.3 ml) was added. Pyridine (4 equiv., 44 mmol, 3.5 ml) was added to the reaction solution. The resulting mixture was stirred at room temperature over night. The solvent was removed and the residue was partitioned between EtOAc and water. The organic phase was collected and washed with 1M HCl and water and finally with brine. Drying (over Na₂SO₄) and removal of the solvent yielded a brown oil. Yield: 91%.

### Step 6:

The product from step 5 (IV) (1 equiv., 10 mmol, 4.86 g) was dissolved in AcOH and NaBO₃ (5 equiv., 50 mmol, 7.7 g) and the resulting mixture was warmed to 50° C. over. The reaction was allowed to proceed over night at 50° C. The solvent was removed in vacuo and water along with EtOAc was added. The organic phase was collected and the aqueous phase was extracted with EtOAc. The combined organic phases were washed with water until the washings were of neutral pH. Drying and evaporation of the solvent resulted in a clear oil. Yield: 88%

### Step 7:

The product from step 6 (VII) was treated in an identical manner as for step 4 using the identical ratio of reagent to substrate. Yield: 85%

### Step 8:

The product from step 7 (VIII) (1 equiv., 11 mmol, 5.6 g) was dissolved in DCM and diethylamine (3 equiv., 33 mmol, 3.4 ml) was added to the solution. Stirring was continued over night at reflux. The solvent was removed and the crude product was dissolved in NaOH (1M). The resulting solution was washed several times with ether and then acidified using 6M HCl. A pale yellow precipitate formed and was collected and allowed to dry under vacuum. The solid was chromatographed using 50% EtOAc in heptane containing 1% AcOH. Yield: 67%

### Step 9:

The compound from step 8 (IX) (typically 1 equiv., 0.305 mmol, 100 mg) was dissolved in dry DMF (2.0 ml). To this solution was added NEM (4 equiv., 1.208 mmol, 0.154 ml) and TBTU (1 equiv., 0.305 mmol, 97 mg). The solution was left standing for 1 h at room temperature. The appropriate amine (typically 1.2 equiv.) was added and the solution was left standing at room temperature over night. The solution was acidified with TFA and directly purified by preparative HPLC.

### Step 10:

The compounds from step 9 (X) (typically 0.1-0.3 mmol) were dissolved in dioxane (1.0 ml) and hydroxylamine (50% in water, 0.25 ml) was added. The solutions were warmed to 50° C. over night. The solutions were acidified with TFA and purified on preparative HPLC to yield the desired products (XI).

### Biological Assays

The compounds of this invention may be tested in the following biological assay in order to determine the concentration of compound (IC₅₀) required for exhibiting the desired pharmacological effect.

### Bacterial Peptide Deformylase (PDF) Assay

The IC₅₀ value of a compound of the invention as a bacterial PDF inhibitor was determined using the following assay.

### Materials:

Assay buffer (pH 7.2): 0.1 M MOPS pH was adjusted to 7.2 with NaOH, containing 0.25 M NaCl, 100 microgram/mL catalase and 1 mg/mL BSA.

Enzyme mix: 670 ng/mL of enzyme (to finally have 50 ng of enzyme per well).

Substrate: 10 mM f-Met-Ala was made up from 200 mM f-Met-Ala in methanol with assay buffer.

TNBS solution: Freshly dilute 1 M TNBS stock solution to 1:10 with water.

Buffer C: 0.5 M borate buffer adjusted to pH 9.5 with NaOH.

Buffer D: 0.2 ml of freshly prepared 0.5 M Na₂SO₃ was mixed with 9.8 mL of 0.5M NaH₂PO₄.

Inhibitor solution: 2 mM Sodium 4-(hydroxymercurio) benzoate in assay buffer.

### Method (Assay Conditions):

The assay was performed in a 96 Microtiter plate containing test compound. To each well containing test compound mix was added 75 microliter of enzyme mix from E. coli followed, by the addition of 25 microliter of substrate mix. The resulting mix was incubated for 30 minutes at room temperature with shaking. TNBS solution (50 microliter/well) was added and the resulting mixture was incubated for 15 minutes under shaking. Buffer C was then added (20 microliter/well). After incubating at room temperature for 15 minutes under shaking, buffer D was added (50 microliter/well). The optical diffraction was then measured at 420 nm, thereby determining the IC₅₀ value.

The assay was repeated using enzyme mix from S. aureus.

The compounds and processes of the invention will be better understood in connection with the following examples, which are intended as an illustration of and not as a limitation upon the scope of the invention.

### Example 1

### 2-(3,4-Dihydro-2H-benzo[1,4]thiazin-2-yl)-N-hydroxy-acetamide

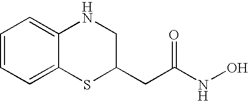

The title compound was prepared according to Method B omitting step 1 in Scheme B.

¹H NMR (MeOD-d₄): δ7.02-6.94 (m, 2H), 6.19-6.76 (m, 2H), 3.85-3.73 (m, 1H), 3.73 (dd, J=12.7, 3.25 Hz, 1H), 3.41 (dd, J=12.2, 6.25 Hz, 1H), 2.55 (dd, J=7.00, 4.25 Hz, 1H).

IC₅₀ (microM): 56.7 (enzyme from E. coli)
37.6 (enzyme from S. aureus)

### Example 2

### 2-(1,1-Dioxo-1,2,3,4-tetrahydro-1λ⁶-benzo[1,4]thiazin-2-yl)-N-hydroxy-acetamide

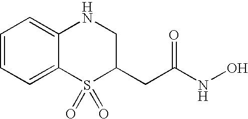

The title compound was prepared according to Method B.

Mass found (M+H): 257.022 Mass calculated (M): 256.05

IC₅₀ (microM): 3.4 (enzyme from E. coli)
1.5 (enzyme from S. aureus).

### Example 3

### 2-(4-Ethyl-3,4-dihydro-2H-benzo[1,4]thiazin-2-yl)-N-hydroxy-acetamide

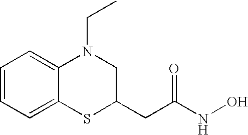

The title compound was prepared according to Method C using acetyl chloride.

Mass found (M+H): 253.153. Mass calculated (M): 252.09

IC₅₀ (microM): 2.6 (enzyme from E. coli)
5.6 (enzyme from S. aureus).

### Example 4

### 2-(4-Ethyl-1,1-dioxo-1,2,3,4-tetrahydro-1λ⁶-benzo[1,4]thiazin-2-yl)-N-hydroxy-acetamide

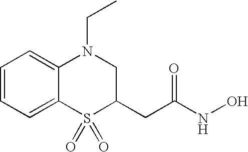

The title compound was prepared according to Method A using acetyl chloride.

¹H NMR (MeOD-d₄): δ7.68 (dd, J=8.25, 2.0 Hz, 1H), 7.44 (ddd, J=7.62, 6.75, 1.75 Hz, 1H), 6.91 (d, J=8.75, 1H), 6.81 (t, J=7.00, 1H), 4.1104.04 (m, 1H), 3.82-3.65 (m, 2H), 3.53 (septet, J=7.5 Hz, 2H), 2.72 (dd, J=15.0, 4.75 Hz, 1H), 2.34 (dd, J=15.0, 9.25 Hz, 1H), 1.21 (t, J=7.0 Hz, 3H).

IC₅₀ (microM): 1.4 (enzyme from E. coli)
2.0 (enzyme from S. aureus).

### Example 5

### N-Hydroxy-2-(4-propyl-3,4-dihydro-2H-benzo[1,4]thiazin-2-yl)-acetamide

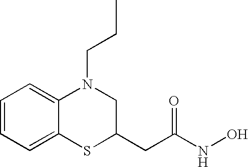

The title compound was prepared according to Method C using propionyl chloride.

Mass found (M+H): 267.160. Mass calculated (M): 266.11

IC₅₀ (microM): 7.0 (enzyme from E. coli)
8.5 (enzyme from S. aureus).

### Example 6

### 2-(1,1-Dioxo-4-propyl-1,2,3,4-tetrahydro-1λ⁶-benzo[1,4]thiazin-2-yl)-N-hydroxy-acetamide

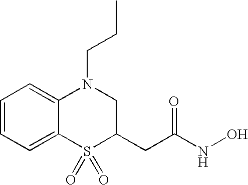

The title compound was prepared according to Method A using propionyl chloride.

Mass found (M+H): 299.070. Mass calculated (M): 298.10

IC₅₀ (microM): 1.6 (enzyme from E. coli)
1.9 (enzyme from S. aureus).

### Example 7

### 2-(4-Butyl-3,4-dihydro-2H-benzo[1,4]thiazin-2-yl)-N-hydroxy-acetamide

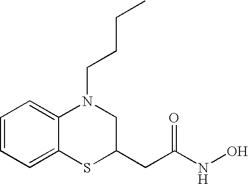

The title compound was prepared according to Method C using butyryl chloride.

Mass found (M+H): 281.168. Mass calculated (M): 280.12

IC₅₀ (microM): 22.0 (enzyme from E. coli)
35.5 (enzyme from S. aureus).

### Example 8

### 2-(4-Butyl-1,1-dioxo-1,2,3,4-tetrahydro-1λ⁶-benzo[1,4]thiazin-2-yl)-N-hydroxy-acetamide

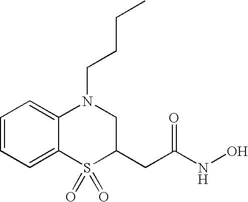

The title compound was prepared according to Method A using butyryl chloride.

Mass found (M+H): 313.082. Mass calculated (M): 312.11

IC₅₀ (microM): 8.8 (enzyme from E. coli)
8.1 (enzyme from S. aureus).

### Example 9

### 2-(4-Benzyl-1,1-dioxo-1,2,3,4-tetrahydro-1λ⁶-benzo[1,4]thiazin-2-yl)-N-hydroxy-acetamide

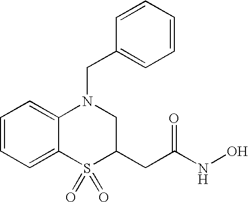

The title compound was prepared according to Method A benzoyl chloride.

¹H NMR (MeOD-d₄): δ7.72 (dd, J=8.25, 1.5 Hz, 1H), 7.39-7.22 (m, 6H), 6.87-6.81 (m, 2H), 4.72 (dd, J=28.5, 17.5 Hz, 2H), 4.20 (dd, J=13.9, 2.75 Hz, 1H), 3.86 (dd, J=13.25, 7.5 Hz, 1H), 3.87-3.78 (m, 1H), 2.78 (dd, J=15.0, 5.0 Hz, 1H), 2.38 (dd, J=15.0, 8.75 Hz, 1H).

IC₅₀ (microM): 21.2 (enzyme from E. coli)
21.4 (enzyme from S. aureus).

### Example 10

### 2-[4-(3-Fluoro-benzyl)-1,1-dioxo-1,2,3,4-tetrahydro-1λ⁶-benzo[1,4]thiazin-2-yl]-N-hydroxy-acetamide

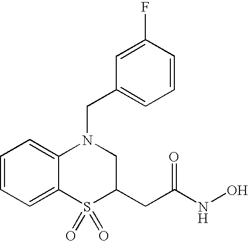

The title compound was prepared according to Method A using 3-fluorobenzoyl chloride.

Mass found (M+H): 365.040. Mass calculated (M): 364.09

IC₅₀ (microM): 27.7 (enzyme from E. coli)
28.4 (enzyme from S. aureus).

### Example 11

### 4-Ethyl-2-hydroxycarbamoylmethyl-1,1-dioxo-1,2,3,4-tetrahydro-1λ⁶-benzo[1,4]thiazine-6-carboxylic acid benzylamide

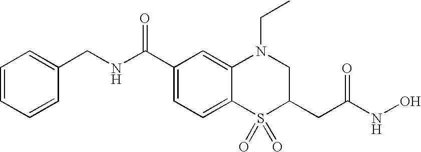

The title compound was prepared according to Method D using benzyl amine.

Mass found (M+H): 418.087. Mass calculated (M): 417.14

IC₅₀ (microM): 17.6 (enzyme from E. coli)
8.9 (enzyme from S. aureus).

### Example 12

### 4-Ethyl-2-hydroxycarbamoylmethyl-1,1-dioxo-1,2,3,4-tetrahydro-1λ⁶-benzo[1,4]thiazine-6-carboxylic acid 2-fluoro-benzylamide

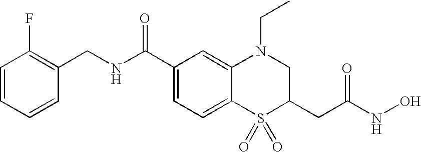

The title compound was prepared according to Method D using 2-fluorobenzyl amine.

Mass found (M+H): 436.134. Mass calculated (M): 435.13

IC₅₀ (microM): 10.8 (enzyme from E. coli)
36.8 (enzyme from S. aureus).

### Example 13

### 4-Ethyl-2-hydroxycarbamoylmethyl-1,1-dioxo-1,2,3,4-tetrahydro-1λ⁶-benzo[1,4]thiazine-6-carboxylic acid 3-fluoro-benzylamide

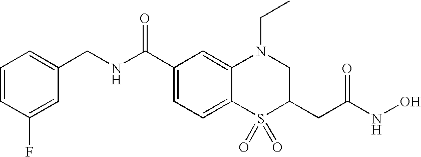

The title compound was prepared according to Method D using 3-fluorobenzyl amine.

Mass found (M+H): 436.315. Mass calculated (M): 435.13

IC₅₀ (microM): 11.7 (enzyme from E. coli)
13.7 (enzyme from S. aureus).

### Example 14

### 4-Ethyl-2-hydroxycarbamoylmethyl-1,1-dioxo-1,2,3,4-tetrahydro-1λ⁶-benzo[1,4]thiazine-6-carboxylic acid 4-fluoro-benzylamide

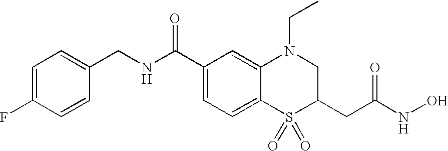

The title compound was prepared according to Method D using 4-fluorobenzyl amine.

Mass found (M+H): 436.251. Mass calculated (M): 435.13

IC₅₀ (microM): 10.5 (enzyme from E. coli)
10.0 (enzyme from S. aureus).

### Example 15

### 4-Ethyl-2-hydroxycarbamoylmethyl-1,1-dioxo-1,2,3,4-tetrahydro-1λ⁶-benzo[1,4]thiazine-6-carboxylic acid 2-bromo-benzylamide

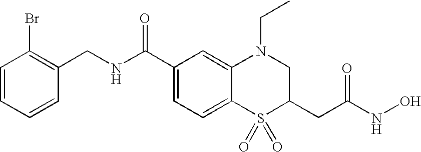

The title compound was prepared according to Method D using 2-bromobenzylamine

Mass found (M+H): 496.145. Mass calculated (M): 495.05

IC₅₀ (microM): 20.0 (enzyme from E. coli)
9.6 (enzyme from S. aureus).

### Example 16

### 4-Ethyl-2-hydroxycarbamoylmethyl-1,1-dioxo-1,2,3,4-tetrahydro-1λ⁶-benzo[1,4]thiazine-6-carboxylic acid 3-bromo-benzylamide

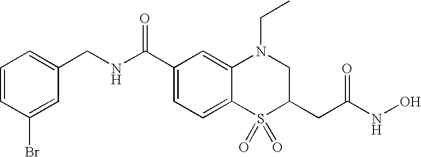

The title compound was prepared according to Method D using 3-bromobenzylamine.

Mass found (M+H): 495.258. Mass calculated (M): 495.05

IC₅₀ (microM): 2.0 (enzyme from E. coli)
9.7 (enzyme from S. aureus).

### Example 17

### 4-Ethyl-2-hydroxycarbamoylmethyl-1,1-dioxo-1,2,3,4-tetrahydro-1λ⁶-benzo[1,4]-thiazine-6-carboxylic acid 4-bromo-benzylamide

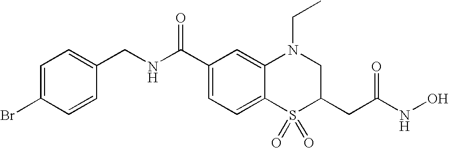

The title compound was prepared according to Method D using 4-bromobenzylamine.

Mass found (M+H): 496.081. Mass calculated (M): 495.05

IC₅₀ (microM): 1.5 (enzyme from E. coli)
3.4 (enzyme from S. aureus).

### Example 18

### 4-Ethyl-2-hydroxycarbamoylmethyl-1,1-dioxo-1,2,3,4-tetrahydro-1λ⁶-benzo[1,4]thiazine-6-carboxylic acid 2-nitro-benzylamide

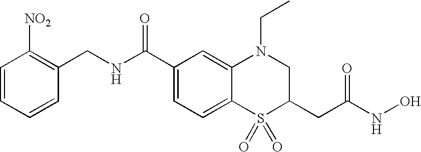

The title compound was prepared according to Method D using 2-nitrobenzylamine.

Mass found (M+H): 463.222. Mass calculated (M): 462.12

IC₅₀ (microM): 17.6 (enzyme from E. coli)
11.3 (enzyme from S. aureus).

### Example 19

### 4-Ethyl-2-hydroxycarbamoylmethyl-1,1-dioxo-1,2,3,4-tetrahydro-1λ⁶-benzo[1,4]thiazine-6-carboxylic acid 3-nitro-benzylamide

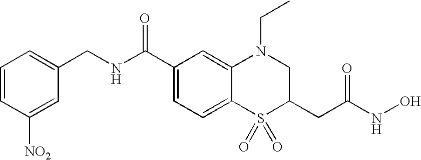

The title compound was prepared according to Method D using 3-nitrobenzylamine.

Mass found (M+H): 463.286. Mass calculated (M): 462.12

IC₅₀ (microM): 3.3 (enzyme from E. coli)
11.9 (enzyme from S. aureus).

### Example 20

### 4-Ethyl-2-hydroxycarbamoylmethyl-1,1-dioxo-1,2,3,4-tetrahydro-1λ⁶-benzo[1,4]thiazine-6-carboxylic acid 4-nitro-benzylamide

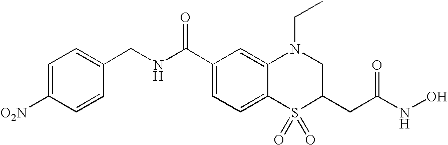

The title compound was prepared according to Method D using 4-nitrobenzylamine.

Mass found (M+H): 463.222. Mass calculated (My: 462.12

IC₅₀ (microM): 1.2 (enzyme from E. coli)
3.9 (enzyme from S. aureus).

### Example 21

### 4-Ethyl-2-hydroxycarbamoylmethyl-1,1-dioxo-1,2,3,4-tetrahydro-1λ⁶-benzo[1,4]thiazine-6-carboxylic acid 2-methoxy-benzylamide

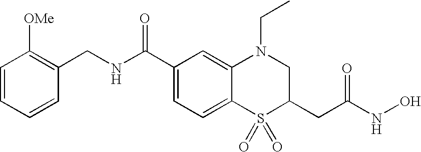

The title compound was prepared according to Method D using 2-methoxybenzylamine.

Mass found (M+H): 448.281. Mass calculated (M): 447.15

IC₅₀ (microM): 1.5 (enzyme from E. coli)
1.5 (enzyme from S. aureus).

### Example 22

### 4-Ethyl-2-hydroxycarbamoylmethyl-1,1-dioxo-1,2,3,4-tetrahydro-1λ⁶-benzo[1,4]thiazine-6-carboxylic acid 3-methoxy-benzylamide

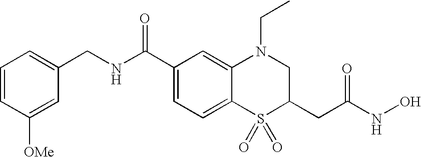

The title compound was prepared according to Method D using 3-methoxybenzylamine

Mass found (M+H): 448.102. Mass calculated (M): 447.15

IC₅₀ (microM): 1.0 (enzyme from E. coli)
2.3 (enzyme from S. aureus).

### Example 23

### 4-Ethyl-2-hydroxycarbamoylmethyl-1,1-dioxo-1,2,3,4-tetrahydro-1λ⁶-benzo[1,4]thiazine-6-carboxylic acid 4-methoxy-benzylamide

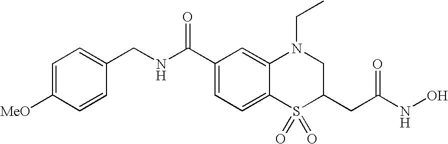

The title compound was prepared according to Method D using 4-methoxybenzylamine.

Mass found (M+H): 448.281. Mass calculated (M): 447.15

IC₅₀ (microM): 4.9 (enzyme from E. coli)
13.1 (enzyme from S. aureus).

### Example 24

### 4-Ethyl-2-hydroxycarbamoylmethyl-1,1-dioxo-1,2,3,4-tetrahydro-1λ⁶-benzo[1,4]thiazine-6-carboxylic acid 3-trifluoromethyl-benzylamide

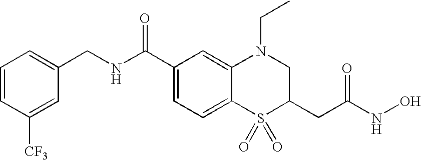

The title compound was prepared according to Method D using 3-trifluoromethylbenzylamine.

Mass found (M+H): 486.268. Mass calculated (M): 485.12

IC₅₀ (microM): 2.0 (enzyme from E. coli)
8.3 (enzyme from S. aureus).

### Example 25

### 4-Ethyl-2-hydroxycarbamoylmethyl-1,1-dioxo-1,2,3,4-tetrahydro-1λ⁶-benzo[1,4]thiazine-6-carboxylic acid 4-trifluoromethyl-benzylamide

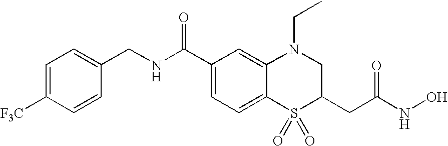

The title compound was prepared according to Method D using 4-trifluoromethylbenzylamine.

Mass found (M+H): 486.205. Mass calculated (M): 485.12

IC₅₀ (microM): <200 (enzyme from E. coli)
4.2 (enzyme from S. aureus).

### Example 26

### 4-Ethyl-2-hydroxycarbamoylmethyl-1,1-dioxo-1,2,3,4-tetrahydro-1λ⁶-benzo[1,4]thiazine-6-carboxylic acid 4-trifluoromethoxybenzylamide

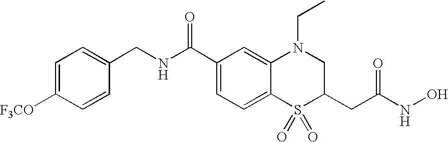

The title compound was prepared according to Method D using 4-trifluoromethoxybenzylamine.

Mass found (M+H): 502.286. Mass calculated (M): 501.12

IC₅₀ (microM): 1.1 (enzyme from E. coli)
5.0 (enzyme from S. aureus).

### Example 27

### 4-Ethyl-2-hydroxycarbamoylmethyl-1,1-dioxo-1,2,3,4-tetrahydro-1λ⁶-benzo[1,4]thiazine-6-carboxylic acid 4-dimethylaminobenzylamide

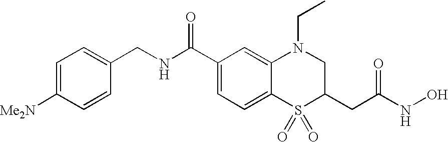

The title compound was prepared according to Method D using 4-dimethylaminobenzylamine.

Mass found (M+H): 461.147. Mass calculated (M): 460.18

IC₅₀ (microM): 9.3 (enzyme from E. coli)
18.2 (enzyme from S. aureus).

### Example 28

### 4-Ethyl-2-hydroxycarbamoylmethyl-1,1-dioxo-1,2,3,4-tetrahydro-1λ⁶-benzo[1,4]thiazine-6-carboxylic acid (pyridin-4-ylmethyl)-amide

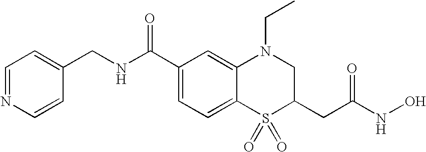

The title compound was prepared according to Method D using 4-pyridylmethylamine.

Mass found (M+H): 419.100. Mass calculated (M): 418.13

IC₅₀ (microM): >200 (enzyme from E. coli)
70.5 (enzyme from S. aureus).

### Example 29

### 4-Ethyl-2-hydroxycarbamoylmethyl-1,1-dioxo-1,2,3,4-tetrahydro-1λ⁶-benzo[1,4]thiazine-6-carboxylic acid (thiophen-2-ylmethyl)-amide

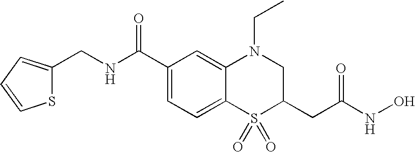

The title compound was prepared according to Method D using 2-thienylmethylamine.

Mass found (M+H): 424.039. Mass calculated (M): 423.09

IC₅₀ (microM): 9.5 (enzyme from E. coli)
3.4 (enzyme from S. aureus).

### Example 30

### 4-Ethyl-2-hydroxycarbamoylmethyl-1,1-dioxo-1,2,3,4-tetrahydro-1λ⁶-benzo[1,4]thiazine-6-carboxylic acid (1H-pyrazol-3-yl)-amide

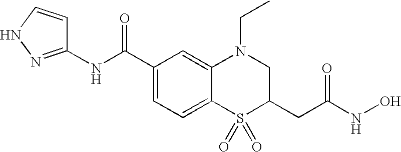

The title compound was prepared according to Method D using 1H-pyrazol-3-ylamine.

Mass found (M+H): 394.152. Mass calculated (M): 393.11

IC₅₀ (microM): 11.5 (enzyme from E. coli)
30.8 (enzyme from S. aureus).

### Example 31

### 4-Ethyl-2-hydroxycarbamoylmethyl-1,1-dioxo-1,2,3,4-tetrahydro-1λ⁶-benzo[1,4]thiazine-6-carboxylic acid thiazol-2-ylamide

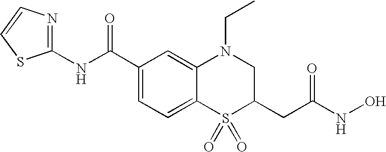

The title compound was prepared according to Method D using thiazol-2-ylamine.

Mass found (M+H): 411.058. Mass calculated (M): 410.07

IC₅₀ (microM): 12.3 (enzyme from E. coli)
20.8 (enzyme from S. aureus).

### Example 32

### 2-[4-Ethyl-6-(4-methyl-piperazine-1-carbonyl)-1,1-dioxo-1,2,3,4-tetrahydro-1λ⁶-benzo[1,4]thiazin-2-yl]-N-hydroxy-acetamide

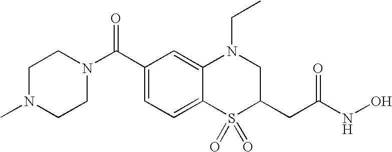

The title compound was prepared according to Method D using N-methylpiperazine.

Mass found (M+H): 411.185. Mass calculated (M): 410.16

IC₅₀ (microM): >200 (enzyme from E. coli)
128.0 (enzyme from S. aureus).

### Example 33

### 4-Ethyl-2-hydroxycarbamoylmethyl-1,1-dioxo-1,2,3,4-tetrahydro-1λ⁶-benzo[1,4]thiazine-6-carboxylic acid (5-thiophen-2-yl-2H-pyrazol-3-yl)-amide

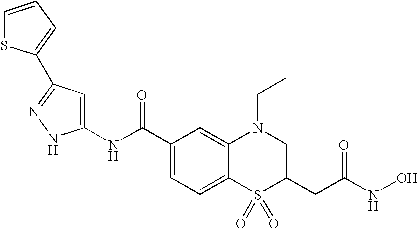

The title compound was prepared according to Method D using 5-thiophen-2-yl-2H-pyrazol-3-ylamine.

Mass found (M+H): 476.028. Mass calculated (M): 475.10

IC₅₀ (microM): 0.9 (enzyme from E. coli)
3.0 (enzyme from S. aureus).

### Example 34

### 4-Ethyl-2-hydroxycarbamoylmethyl-1,1-dioxo-1,2,3,4-tetrahydro-1λ²-benzo[1,4]-thiazine-6-carboxylic acid (8-methyl-8-aza-bicyclo[3.2.1]oct-3-ylamide

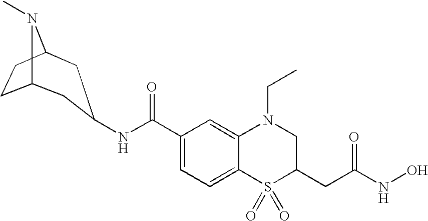

The title compound was prepared according to Method D using 8-methyl-8-aza-bicyclo[3.2.1]oct-3-ylamine.

Mass found (M+H): 451.141. Mass calculated (M): 450.19

IC₅₀ (microM): 13.8 (enzyme from E. coli)
6.0 (enzyme from S. aureus).

### Example 35

### 2-(4-Cyclopropylmethyl-1,1-dioxo-1,2,3,4-tetrahydro-1λ⁶-benzo[1,4]thiazin-2-yl-N-hydroxy-acetamide

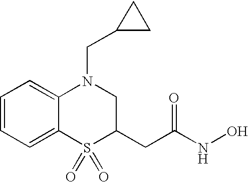

The title compound was prepared according to Method A using cyclopropylcarbonyl chloride.

¹H NMR (MeOD-d₄): δ7.70 (dd, J=7.87, 1.25 Hz, 1H), 7.46 (ddd, J=8.75, 7.0, 2.0 Hz, 1H), 7.02 (d, J=8.25, 1H), 6.84 (t, J=7.00, 1H), 4.18 (dd, J=13.2, 2.75 Hz, 1H), 3.89 (dd, J=13.7, 6.75 Hz, 1H), 3.75-3.66 (m, 1H), 3.49 (dd, J=15.0, 6.5 Hz, 1H), 3.27 (dd, J=15.0, 7.5 Hz, 1H), 2.73 (dd, J=15.0, 4.75 Hz, 1H), 2.36 (dd, J=16.2, 9.25 Hz, 1H), 1.19-1.05 (m, 1H), 0.65-0.57 (m, 2H), 0.38-0.35 (m, 2H).

IC₅₀ (microM): 2.1 (enzyme from E. coli)
4.8 (enzyme from S. aureus).

### Example 36

### 2-(4-Cyclobutylmethyl-1,1-dioxo-1,2,3,4-tetrahydro-1λ⁶-benzo[1,4]thiazin-2-yl)-N-hydroxy-acetamide

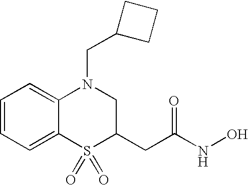

The title compound was prepared according to Method A using cyclobutylcarbonyl chloride.

Mass found (M+H): 325.111. Mass calculated (M): 324.11

IC₅₀ (microM): 3.3 (enzyme from E. coli)
3.8 (enzyme from S. aureus).

### Example 37

### 2-(4-Cyclopentylmethyl-1,1-dioxo-1,2,3,4-tetrahydro-1λ⁶-benzo[1,4]thiazin-2-yl)-N-hydroxy-acetamide

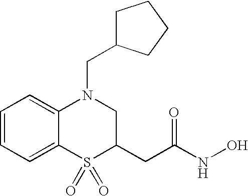

The title compound was prepared according to Method A using cyclopentylcarbonyl chloride.

Mass found (M+H): 339.060. Mass calculated (M): 338.13

IC₅₀ (microM): 11.1 (enzyme from E. coli)
13.9 (enzyme from S. aureus).

### Example 38

### 2-(4-Cyclohexylmethyl-1,1-dioxo-1,2,3,4-tetrahydro-1λ⁶-benzo[1,4]thiazin-2-yl)-N-hydroxy-acetamide

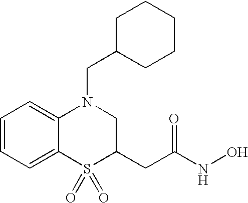

The title compound was prepared according to Method A using cyclohexylcarbonyl chloride.

Mass found (M+H): 353.074. Mass calculated (M): 352.15

IC₅₀ (microM): 33.2 (enzyme from E. coli)
18.1 (enzyme from S. aureus).

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Various references are cited herein, the disclosure of which are incorporated by reference in their entireties.

### Other Aspects of the Invention

Specific embodiments of the invention are:

1. A compound of formula (I)

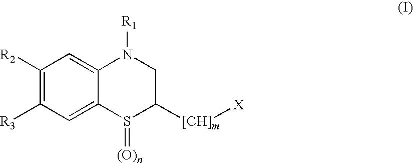

or a pharmaceutically acceptable salt or ester thereof,
wherein

### X is —CONHOH, —COOH or —N(OH)CHO;

n is zero or an integer 1 or 2;
m is an integer 1, 2, 3 or 4;
R₁ is selected from the group consisting of hydrogen, C₁₋₆alkyl, C₂₋₆alkenyl, C₃₋₁₀cycloalkyl, C₁₋₆alkyl-C₃₋₁₀ cycloalkyl, C₃₋₇ heterocycloalkyl, C₁₋₆ alkoxy, C₁₋₆ alkylamino, C₁₋₆ alkylmercapto, C₁₋₆ alkylhydroxy, C₁₋₆ alkylthio, alkylamino-C₁₋₆alkyl, dialkylamino-C₁₋₆ alkyl; and any aryl, heteroaryl, C₁₋₆ alkylaryl or C₁₋₆ alkylheteroaryl optionally substituted with one or more substituents independently selected from halogen, hydroxy, amino, mercapto, nitro, cyano, trifluoromethyl, C₁₋₆ alkyl, C₁₋₆ alkoxy and C₁₋₆ alkylthio;
one of R₂ and R₃ is selected from the group consisting of halogen, hydrogen, carboxylic acid, —CONR₄R₅ and —CONHR₅, in which R₄ and R₅ are identical or different and independently of each other are selected from the group consisting of C₃₋₇ heterocycloalkyl and any of C₁₋₆ alkyl-C₃₋₇heterocycloalkyl, aryl, heteroaryl, C₁₋₆ alkylaryl and C₁₋₆ alkylheteroaryl optionally substituted with one or more substituents independently selected from halogen, hydroxy, amino, mercapto, nitro, cyano, trifluoromethyl, C₁₋₆ alkyl, C₁₋₆ alkoxy, C₁₋₆ alkylthio, C₁₋₆ alkylhydroxy, C₁₋₆alkylamino, alkylamino-C₁₋₆alkyl and dialkylamino-C₁₋₆alkyl; and
the other of R₂ and R₃ is selected from the group consisting of hydrogen, C₁₋₆ alkyl, C₂₋₆ alkenyl, C₃₋₁₀ cycloalkyl, C₁₋₆ alkyl-C₃₋₁₀ cycloalkyl, C₃₋₇ heterocycloalkyl, C₁₋₆ alkoxy, C₁₋₆-alkylamino, C₁₋₆alkylmercapto, C₁₋₆alkylhydroxy, C₁₋₆alkylthio, alkylamino-C₁₋₆alkyl, dialkylamino-C₁₋₆alkyl; and any aryl, heteroaryl, C₁₋₆ alkylaryl or C₁₋₆ alkylheteroaryl optionally substituted with one or more substituents independently selected from halogen, hydroxy, amino, mercapto, nitro, cyano, trifluoromethyl, C₁₋₆ alkyl, C₁₋₆ alkoxy and C₁₋₆ alkylthio.

2. The compound according to item 1, wherein X is —CONHOH.

3. The compound according to item 1, wherein X is —COOH.

4. The compound according to item 1, wherein X is —N(OH)COH.

5. The compound according to item 1, wherein R₁ is selected from the group consisting of hydrogen, C₁₋₆ alkyl, C₃₋₁₀ cycloalkyl, C₁₋₆ alkyl-C₃₋₁₀ cycloalkyl, C₁₋₆ alkylamino, C₁₋₆ alkylhydroxy; and any aryl, C₁₋₆ alkylaryl or C₁₋₆ alkylheteroaryl optionally substituted with one or more substituents independently selected from halogen, hydroxy, amino, mercapto, nitro, cyano, trifluoromethyl, C₁₋₆alkyl, C₁₋₆alkoxy, and C₁₋₆ alkylthio.

6. The compound according to item 1, wherein R₁ is selected from the group consisting of hydrogen, methyl, ethyl, propyl, butyl, cyclopropyl, cyclobutyl, cyclopentyl, methyl cyclopropyl, methyl cyclobutyl, methyl cyclohexyl, ethyl cyclohexyl, ethylamino, propylamino, butylamino, methylhydroxy, ethylhydroxy, propylhydroxy, butylhydroxy, phenyl, benzyl, fluorosubstituted phenyl, fluorosubstituted benzyl, chlorosubstituted phenyl, chlorosubstituted benzyl, bromo substituted phenyl and bromo substituted benzyl.

7. The compound according to item 1, wherein one of R₂ and R₃ is hydrogen, fluorine, chlorine, bromine, iodine or carboxylic acid.

8. The compound according to item 1, wherein one of R₂ and R₃ is —CONHR₅ or —CONR₄R₅.

9. The compound according to item 1, wherein one of R₂ and R₃ is hydrogen or C₃₋₇ heterocycloalkyl; or aryl, heteroaryl, C₁₋₆ alkylaryl or C₁₋₆ alkylheteroaryl optionally substituted with one or more substituents independently selected from halogen, hydroxy, amino, mercapto, nitro, cyano, trifluoromethyl, C₁₋₆ alkyl, C₁₋₆ alkoxy and C₁₋₆ alkylthio.

10. The compound according to item 1, wherein R₄ or R₅ is C₃₋₇heterocycloalkyl, C₁₋₆ alkyl-C₃₋₇ heterocycloalkyl, heteroaryl or C₁₋₆ alkylheteroaryl having one or more heteroatoms selected among N, O and S.

11. The compound according to item 1, wherein R₄ or R₅ is aryl, heteroaryl, C₁₋₆alkylaryl or C₁₋₆ alkylheteroaryl, any of which may be substituted with one or more substituents independently selected from halogen, hydroxy, amino, mercapto, nitro, cyano, trifluoromethyl, C₁₋₆ alkyl, C₁₋₆ alkoxy, C₁₋₆ alkylthio, C₁₋₆ alkylhydroxy, C₁₋₆ alkylamino, alkylamino-C₁₋₆alkyl and dialkylamino-C₁₋₆alkyl.

12. The compound according to item 1, wherein R₄ or R₅ is selected from a group consisting of benzyl; mono-, di-, tri- or tetra-fluoro-substituted benzyl, mono-, di-, tri- or tetra-bromo-substituted benzyl, trifluoromethyl substituted benzyl, trifluoromethoxy substituted benzyl, dimethylamino substituted benzyl, nitro substituted benzyl, 5-thiophen-2-yl-2H-pyrazol-3-yl, 8-methyl-8-aza-bicyclo[3.2.1]oct-3-yl, methylpyridyl, methyl-2-thienyl, 3-pyrazolyl, 2-thiazolyl, 4-methyl-1-piperazinyl.

13. The compound according to item 1, wherein R₃ is selected from a group consisting of hydrogen and 1-piperazinyl.

14. The compound according to item 1 selected from the group consisting of
2-(3,4-Dihydro-2H-benzo[1,4]thiazin-2-yl)-N-hydroxy-acetamide
2-(1,1-Dioxo-1,2,3,4-tetrahydro-1λ⁶-benzo[1,4]thiazin-2-yl)-N-hydroxy-acetamide
2-(4-Ethyl-3,4-dihydro-2H-benzo[1,4]thiazin-2-yl)-N-hydroxy-acetamide
2-(4-Ethyl-1,1-dioxo-1,2,3,4-tetrahydro-1λ⁶-benzo[1,4]thiazin-2-yl)-N-hydroxy-acetamide
N-Hydroxy-2-(4-propyl-3,4-dihydro-2H-benzo[1,4]thiazin-2-yl)-acetamide
2-(1,1-Dioxo-4-propyl-1,2,3,4-tetrahydro-1λ⁶-benzo[1,4]thiazin-2-yl)-N-hydroxy-acetamide
2-(4-Butyl-3,4-dihydro-2H-benzo[1,4]thiazin-2-yl)-N-hydroxy-acetamide
2-(4-Butyl-1,1-dioxo-1,2,3,4-tetrahydro-1λ⁶-benzo[1,4]thiazin-2-yl)-N-hydroxy-acetamide
2-(4-Benzyl-1,1-dioxo-1,2,3,4-tetrahydro-1λ⁶-benzo[1,4]thiazin-2-yl)-N-hydroxy-acetamide
2-[4-(3-Fluoro-benzyl)-1,1-dioxo-1,2,3,4-tetrahydro-1λ⁶-benzo[1,4]thiazin-2-yl]-N-hydroxy-acetamide
4-Ethyl-2-hydroxycarbamoylmethyl-1,1-dioxo-1,2,3,4-tetrahydro-1λ⁶-benzo[1,4]thiazine-6-carboxylic acid benzylamide
4-Ethyl-2-hydroxycarbamoylmethyl-1,1-dioxo-1,2,3,4-tetrahydro-1λ⁶-benzo[1,4]thiazine-6-carboxylic acid 2-fluoro-benzylamide
4-Ethyl-2-hydroxycarbamoylmethyl-1,1-dioxo-1,2,3,4-tetrahydro-1λ⁶-benzo[1,4]thiazine-6-carboxylic acid 3-fluoro-benzylamide
4-Ethyl-2-hydroxycarbamoylmethyl-1,1-dioxo-1,2,3,4-tetrahydro-1λ⁶-benzo[1,4]thiazine-6-carboxylic acid 4-fluoro-benzylamide
4-Ethyl-2-hydroxycarbamoylmethyl-1,1-dioxo-1,2,3,4-tetrahydro-1λ⁶-benzo[1,4]thiazine-6-carboxylic acid 2-bromo-benzylamide
4-Ethyl-2-hydroxycarbamoylmethyl-1,1-dioxo-1,2,3,4-tetrahydro-1λ⁶-benzo[1,4]thiazine-4-carboxylic acid 3-bromo-benzylamide
4-Ethyl-2-hydroxycarbamoylmethyl-1,1-dioxo-1,2,3,4-tetrahydro-1λ⁶-benzo[1,4]thiazine-6-carboxylic acid 4-bromo-benzylamide
4-Ethyl-2-hydroxycarbamoylmethyl-1,1-dioxo-1,2,3,4-tetrahydro-1λ⁶-benzo[1,4]thiazine-6-carboxylic acid 2-nitro-benzylamide
4-Ethyl-2-hydroxycarbamoylmethyl-1,1-dioxo-1,2,3,4-tetrahydro-1λ⁶-benzo[1,4]thiazine-6-carboxylic acid 3-nitro-benzylamide
4-Ethyl-2-hydroxycarbamoylmethyl-1,1-dioxo-1,2,3,4-tetrahydro-1λ⁶-benzo[1,4]thiazine-6-carboxylic acid 4-nitro-benzylamide
4-Ethyl-2-hydroxycarbamoylmethyl-1,1-dioxo-1,2,3,4-tetrahydro-1λ⁶-benzo[1,4]thiazine-6-carboxylic acid 2-methoxy-benzylamide
4-Ethyl-2-hydroxycarbamoylmethyl-1,1-dioxo-1,2,3,4-tetrahydro-1λ⁶-benzo[1,4]thiazine-6-carboxylic acid 3-methoxy-benzylamide
4-Ethyl-2-hydroxycarbamoylmethyl-1,1-dioxo-1,2,3,4-tetrahydro-1λ⁶-benzo[1,4]thiazine-6-carboxylic acid 4-methoxy-benzylamide
4-Ethyl-2-hydroxycarbamoylmethyl-1,1-dioxo-1,2,3,4-tetrahydro-1λ⁶-benzo[1,4]thiazine-6-carboxylic acid 3-trifluoromethyl-benzylamide
4-Ethyl-2-hydroxycarbamoylmethyl-1-dioxo-1,2,3,4-tetrahydro-1λ⁶-benzo[1,4]thiazine-6-carboxylic acid 4-trifluoromethyl-benzylamide
4-Ethyl-2-hydroxycarbamoylmethyl-1,1-dioxo-1,2,3,4-tetrahydro-1λ⁶-benzo[1,4]thiazine-6-carboxylic acid 4-trifluoromethoxybenzylamide
4-Ethyl-2-hydroxycarbamoylmethyl-1,1-dioxo-1,2,3,4-tetrahydro-1λ⁶-benzo[1,4]thiazine-6-carboxylic acid 4-dimethylaminobenzylamide
4-Ethyl-2-hydroxycarbamoylmethyl-1,1-dioxo-1,2,3,4-tetrahydro-1λ⁶-benzo[1,4]thiazine-6-carboxylic acid (pyridin-4-ylmethyl)-amide
4-Ethyl-2-hydroxycarbamoylmethyl-1,1-dioxo-1,2,3,4-tetrahydro-1λ⁶-benzo[1,4]thiazine-6-carboxylic acid (thiophen-2-ylmethyl)-amide
4-Ethyl-2-hydroxycarbamoylmethyl-1,1-dioxo-1,2,3,4-tetrahydro-1λ⁶-benzo[1,4]thiazine-6-carboxylic acid (1H-pyrazol-3-yl)-amide
4-Ethyl-2-hydroxycarbamoylmethyl-1,1-dioxo-1,2,3,4-tetrahydro-1λ⁶-benzo[1,4]thiazine-6-carboxylic acid thiazol-2-ylamide
2-[4-Ethyl-6-(4-methyl-piperazine-1-carbonyl)-1,1-dioxo-1,2,3,4-tetrahydro-1λ⁶-benzo[1,4]thiazin-2-yl]-N-hydroxy-acetamide
4-Ethyl-2-hydroxycarbamoylmethyl-1,1-dioxo-1,2,3,4-tetrahydro-1λ⁶-benzo[1,4]thiazine-6-carboxylic acid (5-thiophen-2-yl-2H-pyrazol-3-yl)amide
4-Ethyl-2-hydroxycarbamoylmethyl-1,1-dioxo-1,2,3,4-tetrahydro-1λ⁶-benzo[1,4]thiazine-6-carboxylic acid (8-methyl-8-aza-bicyclo[3.2.1]oct-3-yl)-amide
2-(4-Cyclopropylmethyl-1,1-dioxo-1,2,3,4-tetrahydro-1λ⁶-benzo[1,4]thiazin-2-yl)-N-hydroxy-acetamide
2-(4-Cyclobutylmethyl-1,1-dioxo-1,2,3,4-tetrahydro-1λ⁶-benzo[1,4]thiazin-2-yl)-N-hydroxy-acetamide
2-(4-Cyclopentylmethyl-1,1-dioxo-1,2,3,4-tetrahydro-1λ⁶-benzo[1,4]thiazin-2-yl)-N-hydroxy-acetamide, and
2-(4-Cyclohexylmethyl-1,1-dioxo-1,2,3,4-tetrahydro-1λ⁶-benzo[1,4]thiazin-2-yl)-N-hydroxy-acetamide.

15. The compound according to item 1, which exhibits an IC₅₀ value of less than 500 μM, preferably less than 100 μM, more preferably less than 50 μM, even more preferably less than 1 μM, especially less than 500 nM, particularly less than 100 nM.

16. A pharmaceutical composition comprising, as an active ingredient, a compound according to any of the preceding items or a pharmaceutically acceptable salt thereof together with a pharmaceutically acceptable carrier or diluent.

17. The composition according to item 16 comprising a second active ingredient having antibacterial activity.

18. The composition according to item 16 in unit dosage form, comprising from about 0.05 to about 500 mg, preferably from about 0.1 to about 100 mg, more preferably from about 0.1 to about 50 mg of the compound according to item 1 or a pharmaceutically acceptable salt or ester thereof.

19. A pharmaceutical composition for treatment of infections, the composition comprising, as an active ingredient, a compound according to item 1 or a pharmaceutically acceptable salt thereof together with a pharmaceutically acceptable carrier or diluent.

20. The pharmaceutical composition according to item 19 for the treatment of bacterial infections fully or partly caused by an organism belonging to any of the genera Staphylococcus, Enterococcus, Streptococcus, Haemophilus, Moraxella, Escherichia, Mycobacteria, Mycoplasma, Pseudomonas, Chlamydia, Rickettsia, Klebsiella, Shigella, Salmonella, Bordetella, Clostridia, Helicobacter, Campylobacter, Legionella and Neisseria.

21. The pharmaceutical composition according to any of the items 16, 17, 18, 19 and 20 for oral, nasal, transdermal, pulmonal or parenteral administration.

22. A method for the treatment of ailments, the method comprising administering to a subject in need thereof an effective amount of a compound according to item 1 or a pharmaceutically acceptable salt thereof, or of a composition according to any of the items 16, 17, 18, 19, 20 and 21.

23. The method according to item 22, wherein the effective amount of the compound according to item 1 or a pharmaceutically acceptable salt or ester thereof is in the range of from about 0.05 to about 100 mg per day, preferably from about 0.1 to about 50 mg per day.

24. Use of a compound according to item 1 or a pharmaceutically acceptable salt thereof for the preparation of a medicament.

25. Use of a compound according to item 1 or a pharmaceutically acceptable salt thereof for the preparation of a medicament for treatment of bacterial infections.

26. Use of a compound according to item 1 or a pharmaceutically acceptable salt thereof for the preparation of a medicament for treatment of an infection fully or partly caused by an organism belonging to the group consisting of Staphylococcus, Enterococcus, Streptococcus, Haemophilus, Moraxella, Escherichia, Mycobacteria, Mycoplasma, Pseudomonas, Chlamydia, Rickettsia, Klebsiella, Shigella, Salmonella, Bordetella, Clostridia, Helicobacter, Campylobacter, Legionella and Neisseria.

27. Use of a compound according to item 1 or a pharmaceutically acceptable salt thereof for the preparation of a medicament for treatment of an infection fully or partly caused by an organism belonging to the group consisting of Staphylococcus aureus, Staphylococcus epidermidis, Enterococcus faecium, Enterococcus faecalis, Streptococcus pneumoniae, Haemophilus influenzae, Moraxella catarrhalis, Escherichia coli, Mycobacterium tuberculosis, Mycobacterium ranae, Mycoplasma pneumoniae, Pseudomonas aeruginosa, Chlamydia, Rickettsiae, Klebsiella pneumoniae, Shigella flexneri, Salmonella typhimudum, Bordetella pertussis, Clostridia perfringens, Helicobacter pylori, Campylobacter jejuni, Legionella pneumophila and Neisseria gonorrhoeae.

### LITERATURE LIST

Adams, J. M.; Capecchi, M. R. Proc. Natl. Acad. Sci., USA, 1966, 55, 147-155.
Adams, J. M. J. Mol. Biol. 1968, 33, 571-589.
Clements, J. M.; Ayscough, A. P.; Keavey, K.; East, S. P. Curr. Med. Chem. Anti-Infective Agents 2002, 1, 239-249.
Gennaro, A. R.; Gennaro A. L. Remington, The Science and Practice of Pharmacy, 19th ed., Mack Publishing Co., Easton, Pa., 1995.
Giglione, C.; Pierre, M.; Meinnel, T. Mol. Microbiol. 2000, 36, 1197-1205.
Giglione, C.; Meinnel, T. Emerg. Ther. Targets 2001, 5, 41-57.
Green, T. W. Protective Groups In Organic Synthesis, John Wiley & Sons, New York, 1981.
Pei, D. Emerg. Ther. Targets 2001, 5, 23-40.
Yuan, Z.; Trias, J.; White, R. J. Drug Discov. Today 2001, 6, 954-961.

1. A compound of formula (I) 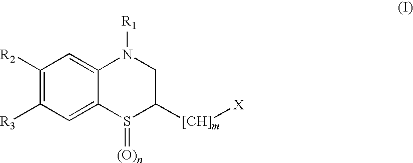
or a pharmaceutically acceptable salt or ester thereof,
wherein
X is —CONHOH, —COOH or —N(OH)CHO;
n is zero or an integer 1 or 2;
m is an integer 1, 2, 3 or 4;
R₁ is selected from the group consisting of hydrogen, C₁₋₆ alkyl, C₂₋₆ alkenyl, C₃₋₁₀ cycloalkyl, C₁₋₆ alkyl-C₃₋₁₀ cycloalkyl, C₃₋₇ heterocycloalkyl, C₁₋₆ alkoxy, C₁₋₆ alkylamino, C₁₋₆ alkylmercapto, C₁₋₆ alkylhydroxy, thioC₁₋₆ alkyl, alkylamino-C₁₋₆alkyl, dialkylamino-C₁₋₆alkyl, an unsubstituted or substituted aryl group, an unsubstituted or substituted heteroaryl group, an unsubstituted or substituted C₁₋₆ alkylaryl group, and an unsubstituted or substituted C₁₋₆ alkylheteroaryl group; wherein a substituted group is substituted with one, two or three substituents independently selected from halogen, hydroxy, amino, mercapto, nitro, cyano, trifluoromethyl, C₁₋₆ alkyl, C₁₋₆ alkoxy and thioC₁₋₆ alkyl;
one of R₂ and R₃ is selected from the group consisting of halogen, hydrogen, carboxylic acid, —CONR₄R₅ and —CONHR₅, in which R₄ and R₅ are identical or different and independently of each other are selected from the group consisting of C₃₋₇ heterocycloalkyl, an unsubstituted or substituted aryl group, an unsubstituted or substituted heteroaryl group, an unsubstituted or substituted C₁₋₆ alkylaryl group, an unsubstituted or substituted C₁₋₆ alkylheteroaryl group and an unsubstituted or substituted C₁₋₆ alkyl-C₃₋₇ heterocycloalkyl group; wherein a substituted group is substituted with one, two or three substituents independently selected from halogen, hydroxy, amino, mercapto, nitro, cyano, trifluoromethyl, C₁₋₆ alkyl, C₁₋₆alkoxy, thioC₁₋₆ alkyl, C₁₋₆alkylhydroxy, C₁₋₆alkylamino, alkylamino-C₁₋₆alkyl and dialkylamino-C₁₋₆alkyl; and
the other of R₂ and R₃ is selected from the group consisting of hydrogen, C₁₋₆ alkyl, C₂₋₆ alkenyl, C₃₋₁₀ cycloalkyl, C₁₋₆ alkyl-C₃₋₁₀ cycloalkyl, C₃₋₇ heterocycloalkyl, C₁₋₆ alkoxy, C₁₋₆ alkylamino, C₁₋₆alkylmercapto, C₁₋₆ alkylhydroxy, thioC₁₋₆alkyl, alkylamino-C₁₋₆alkyl, dialkylamino-C₁₋₆alkyl, an unsubstituted or substituted aryl group, an unsubstituted or substituted heteroaryl group, an unsubstituted or substituted C₁₋₆ alkylaryl group, and an unsubstituted or substituted C₁₋₆ alkylheteroaryl group; wherein a substituted group is substituted with one, two or three substituents independently selected from halogen, hydroxy, amino, mercapto, nitro, cyano, trifluoromethyl, C₁₋₆ alkyl, C₁₋₆ alkoxy and thioC₁₋₆ alkyl.

2. A compound according to claim 1, wherein X is —CONHOH.

3. A compound according to claim 1, wherein X is —COOH.

4. A compound according to claim 1, wherein X is —N(OH)CHO.

5. A compound according to claim 1, wherein R₁ is selected from the group consisting of hydrogen, C₁₋₆alkyl, C₁₋₆ alkyl-C₃₋₁₀ cycloalkyl, C₁₋₆ alkylamino, C₁₋₆ alkylhydroxy, an unsubstituted or substituted C₁₋₆ alkylaryl group, and an unsubstituted or substituted C₁₋₆ alkylheteroaryl group.

6. A compound according to claim 1, wherein R₁ is selected from the group consisting of hydrogen, methyl, ethyl, propyl, butyl, pentyl, methyl cyclopropyl, methyl cyclobutyl, methyl cyclopentyl, methyl cyclohexyl, ethyl cyclohexyl, ethylamino, propylamino, butylamino, methylhydroxy, ethylhydroxy, propylhydroxy, butylhydroxy, benzyl, fluorosubstituted benzyl, chlorosubstituted benzyl, and bromo substituted benzyl.

7. A compound according to claim 1, wherein R₁ is selected from the group consisting of hydrogen, ethyl, propyl, butyl, methyl cyclopropyl, methyl cyclobutyl, methyl cyclopentyl, methyl cyclohexyl, benzyl, and 3-fluorobenzyl.

8. A compound according to claim 1, wherein one of R₂ and R₃ is hydrogen, fluorine, chlorine, bromine, iodine or carboxylic acid.

9. A compound according to claim 1, wherein one of R₂ and R₃ is —CONHR₅ or —CONR₄R₅.

10. A compound according to claim 1, wherein one of R₂ and R₃ is hydrogen or C₃₋₇ heterocycloalkyl, an unsubstituted or substituted aryl group, an unsubstituted or substituted heteroaryl group, an unsubstituted or substituted C₁₋₆ alkylaryl group, and an unsubstituted or substituted C₁₋₆ alkylheteroaryl group.

11. A compound according to claim 1, wherein R₄ or R₅ is C₃₋₇ heterocycloalkyl, C₁₋₆ alkyl-C₃₋₇ heterocycloalkyl, heteroaryl, or C₁₋₆ alkylheteroaryl having one or more heteroatoms selected from N, O, and S.

12. A compound according to claim 1, wherein R₄ or R₅ is an unsubstituted or substituted aryl group, an unsubstituted or substituted heteroaryl group, an unsubstituted or substituted C₁₋₆ alkylaryl group, and an unsubstituted or substituted C₁₋₆ alkylheteroaryl group.

13. A compound according to claim 1, wherein R₄ or R₅ is selected from a group consisting of benzyl; mono-, di-, or tri-fluoro-substituted benzyl, mono-, di-, or tri-bromo-substituted benzyl, methoxy substituted benzyl, trifluoromethyl substituted benzyl, trifluoromethoxy substituted benzyl, dimethylamino substituted benzyl, nitro substituted benzyl, 5-thiophen-2-yl-2H-pyrazol-3-yl, 8-methyl-8-aza-bicyclo[3.2.1]oct-3-yl, methylpyridyl, methyl-2-thienyl, 3-pyrazolyl, 2-thiazolyl, 4-methyl-1-piperazinyl.

14. A compound according to claim 1, wherein R₃ is selected from a group consisting of hydrogen and 1-piperazinyl.

15. A compound according to any claim 1 selected from the group consisting of
2-(3,4-Dihydro-2H-benzo[1,4]thiazin-2-yl)-N-hydroxy-acetamide
2-(1,1-Dioxo-1,2,3,4-tetrahydro-1λ⁶-benzo[1,4]thiazin-2-yl)-N-hydroxy-acetamide
2-(4-Ethyl-3,4-dihydro-2H-benzo[1,4]thiazin-2-yl)-N-hydroxy-acetamide
2-(4-Ethyl-1,1-dioxo-1,2,3,4-tetrahydro-1λ⁶-benzo[1,4]thiazin-2-yl)-N-hydroxy-acetamide
N-Hydroxy-2-(4-propyl-3,4-dihydro-2H-benzo[1,4]thiazin-2-yl)-acetamide
2-(1,1-Dioxo-4-propyl-1,2,3,4-tetrahydro-1λ⁶-benzo[1,4]thiazin-2-yl)-N-hydroxy-acetamide
2-(4-Butyl-3,4-dihydro-2H-benzo[1,4]thiazin-2-yl)-N-hydroxy-acetamide
2-(4-Butyl-1,1-dioxo-1,2,3,4-tetrahydro-1,6-benzo[1,4]thiazin-2-yl)-N-hydroxy-acetamide
2-(4-Benzyl-1,1-dioxo-1,2,3,4-tetrahydro-1λ⁶-benzo[1,4]thiazin-2-yl)-N-hydroxy-acetamide
2-[4-(3-Fluoro-benzyl)-1,1-dioxo-1,2,3,4-tetrahydro-1λ⁶-benzo[1,4]thiazin-2-yl]-N-hydroxy-acetamide
4-Ethyl-2-hydroxycarbamoylmethyl-1,1-dioxo-1,2,3,4-tetrahydro-1λ⁶-benzo[1,4]thiazine-6-carboxylic acid benzylamide
4-Ethyl-2-hydroxycarbamoylmethyl-1,1-dioxo-1,2,3,4-tetrahydro-1λ⁶-benzo[1,4]thiazine-6-carboxylic acid 2-fluoro-benzylamide
4-Ethyl-2-hydroxycarbamoylmethyl-1,1-dioxo-1,2,3,4-tetrahydro-1λ⁶-benzo[1,4]thiazine-6-carboxylic acid 3-fluoro-benzylamide
4-Ethyl-2-hydroxycarbamoylmethyl-1,1-dioxo-1,2,3,4-tetrahydro-1λ⁶-benzo[1,4]thiazine-6-carboxylic acid 4-fluoro-benzylamide
4-Ethyl-2-hydroxycarbamoylmethyl-1,1-dioxo-1,2,3,4-tetrahydro-1λ⁶-benzo[1,4]thiazine-6-carboxylic acid 2-bromo-benzylamide
4-Ethyl-2-hydroxycarbamoylmethyl-1,1-dioxo-1,2,3,4-tetrahydro-1λ⁶-benzo[1,4]thiazine-6-carboxylic acid 3-bromo-benzylamide
4-Ethyl-2-hydroxycarbamoylmethyl-1,1-dioxo-1,2,3,4-tetrahydro-1λ⁶-benzo[1,4]thiazine-6-carboxylic acid 4-bromo-benzylamide
4-Ethyl-2-hydroxycarbamoylmethyl-1,1-dioxo-1,2,3,4-tetrahydro-1λ⁶-benzo[1,4]thiazine-6-carboxylic acid 2-nitro-benzylamide
4-Ethyl-2-hydroxycarbamoylmethyl-1,1-dioxo-1,2,3,4-tetrahydro-1λ⁶-benzo[1,4]thiazine-6-carboxylic acid 3-nitro-benzylamide
4-Ethyl-2-hydroxycarbamoylmethyl-1,1-dioxo-1,2,3,4-tetrahydro-1λ⁶-benzo[1,4]thiazine-6-carboxylic acid 4-nitro-benzylamide
4-Ethyl-2-hydroxycarbamoylmethyl-1,1-dioxo-1,2,3,4-tetrahydro-1λ⁶-benzo[1,4]thiazine-6-carboxylic acid 2-methoxy-benzylamide
4-Ethyl-2-hydroxycarbamoylmethyl-1,1-dioxo-1,2,3,4-tetrahydro-1λ⁶-benzo[1,4]thiazine-6-carboxylic acid 3-methoxy-benzylamide
4-Ethyl-2-hydroxycarbamoylmethyl-1,1-dioxo-1,2,3,4-tetrahydro-1λ⁶-benzo[1,4]thiazine-6-carboxylic acid 4-methoxy-benzylamide
4-Ethyl-2-hydroxycarbamoylmethyl-1,1-dioxo-1,2,3,4-tetrahydro-1λ⁶-benzo[1,4]thiazine-6-carboxylic acid 3-trifluoromethyl-benzylamide
4-Ethyl-2-hydroxycarbamoylmethyl-1,1-dioxo-1,2,3,4-tetrahydro-1λ⁶-benzo[1,4]thiazine-6-carboxylic acid 4-trifluoromethyl-benzylamide
4-Ethyl-2-hydroxycarbamoylmethyl-1,1-dioxo-1,2,3,4-tetrahydro-1λ⁶-benzo[1,4]thiazine-6-carboxylic acid 4-trifluoromethoxybenzylamide
4-Ethyl-2-hydroxycarbamoylmethyl-1,1-dioxo-1,2,3,4-tetrahydro-1λ⁶-benzo[1,4]thiazine-6-carboxylic acid 4-dimethylaminobenzylamide
4-Ethyl-2-hydroxycarbamoylmethyl-1,1-dioxo-1,2,3,4-tetrahydro-1λ⁶-benzo[1,4]thiazine-6-carboxylic acid (pyridin-4-ylmethyl)-amide
4-Ethyl-2-hydroxycarbamoylmethyl-1,1-dioxo-1,2,3,4-tetrahydro-1λ⁶-benzo[1,4]thiazine-6-carboxylic acid (thiophen-2-ylmethyl)-amide
4-Ethyl-2-hydroxycarbamoylmethyl-1,1-dioxo-1,2,3,4-tetrahydro-1λ⁶-benzo[1,4]thiazine-6-carboxylic acid (1H-pyrazol-3-yl)-amide
4-Ethyl-2-hydroxycarbamoylmethyl-1,1-dioxo-1,2,3,4-tetrahydro-1λ⁶-benzo[1,4]thiazine-6-carboxylic acid thiazol-2-ylamide
2-[4-Ethyl-6-(4-methyl-piperazine-1-carbonyl)-1,1-dioxo-1,2,3,4-tetrahydro-1λ⁶benzo[1,4]thiazin-2-yl]-N-hydroxy-acetamide
4-Ethyl-2-hydroxycarbamoylmethyl-1,1-dioxo-1,2,3,4-tetrahydro-1λ⁶-benzo[1,4]thiazine-6-carboxylic acid (5-thiophen-2-yl-2H-pyrazol-3-yl)-amide
4-Ethyl-2-hydroxycarbamoylmethyl-1,1-dioxo-1,2,3,4-tetrahydro-1λ⁶-benzo[1,4]thiazine-6-carboxylic acid (8-methyl-8-aza-bicyclo[3.2.1]oct-3-yl)-amide
2-(4-Cyclopropylmethyl-1,1-dioxo-1,2,3,4-tetrahydro-1λ⁶-benzo[1,4]thiazin-2-yl)-N-hydroxy-acetamide
2-(4-Cyclobutylmethyl-1,1-dioxo-1,2,3,4-tetrahydro-1λ⁶-benzo[1,4]thiazin-2-yl)-N-hydroxy-acetamide
2-(4-Cyclopentylmethyl-1,1-dioxo-1,2,3,4-tetrahydro-1λ⁶-benzo[1,4]thiazin-2-yl)-N-hydroxy-acetamide, and
2-(4-Cyclohexylmethyl-1,1-dioxo-1,2,3,4-tetrahydro-1λ⁶-benzo[1,4]thiazin-2-yl)-N-hydroxy-acetamide.

16. A compound according to claim 1, which in the PDF assay exhibits an IC₅₀ value of less than 500 μM.

17-20. (canceled)

21. A pharmaceutical composition comprising, as an active ingredient, a compound according to claim 1 or a pharmaceutically acceptable salt thereof together with a pharmaceutically acceptable carrier or diluent.

22. A pharmaceutical composition according to claim 21 comprising a second active ingredient having antibacterial activity.

23. A pharmaceutical composition according to any of claims 21 in unit dosage form, comprising from about 1 μg to about 1000 mg of the compound or a pharmaceutically acceptable salt or ester thereof.

24-25. (canceled)

26. A pharmaceutical composition according to claim 21, for oral, nasal, transdermal, pulmonal or parenteral administration.

27. A method for the treatment of ailments, the method comprising administering to a subject in need thereof an effective amount of a compound according to claim 1 or a pharmaceutically acceptable salt thereof.

28. A method according to claim 27, wherein the effective amount of the compound is in the range of from about 1 μg to about 1000 mg.

29-32. (canceled)

33. A method according to claim 27 wherein the subject is suffering from an infection.

34. A method according to claim 27 wherein the subject is suffering from an infection fully or partly caused by an organism belonging to the group consisting of Staphylococcus, Enterococcus, Streptococcus, Haemophilus, Moraxella, Escherichia, Mycobacteria, Mycoplasma, Pseudomonas, Chlamydia, Rickettsia, Klebsiella, Shigella, Salmonella, Bordetella, Clostridia, Helicobacter, Campylobacter, Legionella and Neisseria.

35. A method according to claim 27 wherein the subject is suffering from an infection fully or partly caused by an organism belonging to the group consisting of Staphylococcus aureus, Staphylococcus epidermidis, Enterococcus faecium, Enterococcus faecalis, Streptococcus pneumoniae, Haemophilus influenzae, Moraxella catarrhalis, Escherichia coli, Mycobacterium tuberculosis, Mycobacterium ranae, Mycoplasma pneumoniae, Pseudomonas aeruginosa, Chlamydia, Rickettsiae, Klebsiella pneumoniae, Shigella flexneri, Salmonella typhimurium, Bordetella pertussis, Clostridia perfringens, Helicobacter pylori, Campylobacter jejuni, Legionella pneumophila and Neisseria gonorrhoeae.

36. A method for treating a subject suffering from or susceptible to an infection, comprising administering to the subject an effective amount of a compound of claim 1.

37. A method according to claim 36 wherein the subject is suffering from an infection fully or partly caused by an organism belonging to the group consisting of Staphylococcus, Enterococcus, Streptococcus, Haemophilus, Moraxella, Escherichia, Mycobacteria, Mycoplasma, Pseudomonas, Chlamydia, Rickettsia, Klebsiella, Shigella, Salmonella, Bordetella, Clostridia, Helicobacter, Campylobacter, Legionella and Neisseria.

38. A method according to claim 36 wherein the subject is suffering from an infection fully or partly caused by an organism belonging to the group consisting of Staphylococcus aureus, Staphylococcus epidermidis, Enterococcus faecium, Enterococcus faecalis, Streptococcus pneumoniae, Haemophilus influenzae, Moraxella catarrhalis, Escherichia coli, Mycobacterium tuberculosis, Mycobacterium ranae, Mycoplasma pneumoniae, Pseudomonas aeruginosa, Chlamydia, Rickettsiae, Klebsiella pneumoniae, Shigella flexneri, Salmonella typhimurium, Bordetella pertussis, Clostridia perfringens, Helicobacter pylori, Campylobacter jejuni, Legionella pneumophila and Neisseria gonorrhoeae.
